SEQUENCE LISTING (38 sequences)

SEQ ID NO: 1 - DNA, 3891 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagataggaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 4395 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 4031 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgacaagcttccaccatggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgagaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 3889 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgtccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 3846 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgccaccatgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 6 - DNA, 3969 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatggttacttttgccacgatcttacaggtgaacaaggtgatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccagtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcctgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 7 - DNA, 3765 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatggctttcacagagcattcaccgctgacccctcaccgtcgggacctctgtagccgctctatctggctagcaaggaagattcgttcagacctgactgctcttacggaatcctatgtgaagcatcagggcctgaacaagaacatcaacctggactctgcggatgggatgccagtggcaagcactgatcagtggagtgagctgaccgaggcagagcgactccaagagaaccttcaagcttatcgtaccttccatgttttgttggccaggctcttagaagaccagcaggtgcattttaccccaaccgaaggtgacttccatcaagctatacatacccttcttctccaagtcgctgcctttgcataccagatagaggagttaatgatactcctggaatacaagatcccccgcaatgaggctgatgggatgcctattaatgttggagatggtggtctctttgagaagaagctgtggggcctaaaggtgctgcaggagctttcacagtggacagtaaggtccatccatgaccttcgtttcatttcttctcatcagactgggatcccagcacgtgggagccattatattgctaacaacaagaaaatgtaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 8 - DNA, 3639 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 9 - DNA, 36 bp, Artificial Sequence
  Oligonucleotide BDNF_F
ggatccgccaccatgaccatccttttccttactatg

SEQ ID NO: 10 - DNA, 31 bp, Artificial Sequence
  Oligonucleotide BDNF_R
agggaattcctatcttccccttttaatggtc

SEQ ID NO: 11 - DNA, 20 bp, Artificial Sequence
  Oligonucleotide BDNF_SF
tttggttgcatgaaggctgc

SEQ ID NO: 12 - DNA, 20 bp, Artificial Sequence
  Oligonucleotide BDNF_SR
gccgaactttctggtcctca

SEQ ID NO: 13 - DNA, 38 bp, Artificial Sequence
  Oligonucleotide VEGFA_F
gggggatccaccatgacggacagacagacagacaccgc

SEQ ID NO: 14 - DNA, 37 bp, Artificial Sequence
  Oligonucleotide VEGFA_R
tttggatccaccatgaactttctgctgtcttgggtgc

SEQ ID NO: 15 - DNA, 20 bp, Artificial Sequence
  Oligonucleotide VEGFA_SF
tctgctgtcttgggtgcatt

SEQ ID NO: 16 - DNA, 20 bp, Artificial Sequence
  Oligonucleotide VEGFA_SR
ccagggtctcgattggatgg

SEQ ID NO: 17 - DNA, 40 bp, Artificial Sequence
  Oligonucleotide BFGF_F
gaggaagcttccaccatggtgggtgtggggggtggagatg

SEQ ID NO: 18 - DNA, 35 bp, Artificial Sequence
  Oligonucleotide BFGF_R
gagggaattctcagctcttagcagacattggaaga

SEQ ID NO: 19 - DNA, 20 bp, Artificial Sequence
  Oligonucleotide BFGF_SF
tgtgctaaccgttacctggc

SEQ ID NO: 20 - DNA, 20 bp, Artificial Sequence
  Oligonucleotide BFGF_SR
actgcccagttcgtttcagt

SEQ ID NO: 21 - DNA, 40 bp, Artificial Sequence
  Oligonucleotide NGF_F
tttgtcgaccaccatgtccatgttgttctacactctgatc

SEQ ID NO: 22 - DNA, 32 bp, Artificial Sequence
  Oligonucleotide NGF_R
aatggtacctcaggctcttctcacagccttcc

SEQ ID NO: 23 - DNA, 20 bp, Artificial Sequence
  Oligonucleotide NGF_SF
tgaagctgcagacactcagg

SEQ ID NO: 24 - DNA, 92 bp, Artificial Sequence
  Oligonucleotide NGF_SR
ctcccaacaccatcacctccgggggatccaccatgcagtctttgcctaacagcaatggtttaagctttcagatacatccacaccttttagcg

SEQ ID NO: 25 - DNA, 20 bp, Artificial Sequence
  Oligonucleotide GDNF_SF
gtcactgacttgggtctggg

SEQ ID NO: 26 - DNA, 20 bp, Artificial Sequence
  Oligonucleotide GDNF_SR
gcctgccctactttgtcact

SEQ ID NO: 27 - DNA, 31 bp, Artificial Sequence
  Oligonucleotide NT3_F
aggatccaccatggttacttttgccacgatc

SEQ ID NO: 28 - DNA, 31 bp, Artificial Sequence
  Oligonucleotide NT3_R
tataagctttcatgttcttccgatttttctc

SEQ ID NO: 29 - DNA, 20 bp, Artificial Sequence
  Oligonucleotide NT3_SF
aactgctgcgacaacagaga

SEQ ID NO: 30 - DNA, 20 bp, Artificial Sequence
  Oligonucleotide NT3_SR
gtactcccctcggtgactct

SEQ ID NO: 31 - DNA, 36 bp, Artificial Sequence
  Oligonucleotide CNTF_F
tttgtcgaccaccatggctttcacagagcattcacc

SEQ ID NO: 32 - DNA, 39 bp, Artificial Sequence
  Oligonucleotide CNTF_R
aatggtacctacattttcttgttgttagcaatataatgg

SEQ ID NO: 33 - DNA, 20 bp, Artificial Sequence
  Oligonucleotide CNTF_SF
acatcaacctggactctgcg

SEQ ID NO: 34 - DNA, 20 bp, Artificial Sequence
  Oligonucleotide CNTF_SR
tggaagtcaccttcggttgg

SEQ ID NO: 35 - DNA, 36 bp, Artificial Sequence
  Oligonucleotide IGF1_F
tttgtcgaccaccatgggaaaaatcagcagtcttcc

SEQ ID NO: 36 - DNA, 35 bp, Artificial Sequence
  Oligonucleotide IGF1_R
aatggtacctacttgcgttcttcaaatgtacttcc

SEQ ID NO: 37 - DNA, 20 bp, Artificial Sequence
  Oligonucleotide IGF1_SF
ccatgtcctcctcgcatctc

SEQ ID NO: 38 - DNA, 20 bp, Artificial Sequence
  Oligonucleotide IGF1_SR
accctgtgggcttgttgaaa

(19) United States
(12) Patent Application Publication
(10) Pub. No.: US 2024/0325569 A1
(43) Pub. Date: Oct. 3, 2024

(54) Gene therapy DNA vector based on gene therapy DNA vector VTvaf17 carrying the therapeutic gene selected from the group of BDNF, VEGFA, BFGF, NGF, GDNF, NT3, CNTF, and IGF1 genes for increasing the expression level of these therapeutic genes, method of its production and use, Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli str

(71) Applicants: CELL AND GENE THERAPY LTD, Rostov-on-Don (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/268,731

(22) PCT Filed: Dec. 18, 2019
(86) PCT No.: PCT/RU2019/000967

§ 371 Date: Jun. 21, 2023

(30) Foreign Application Priority Data
Dec. 21, 2018 (RU) 2018145693

(51) Int. Cl. A61K 48/00; A61K 38/18; A61K 38/30; C12N 15/10; C12N 15/85

(52) U.S. Cl. CPC A61K 48/005; A61K 38/185; A61K 38/1866; A61K 38/30; C12N 15/1096; C12N 15/85

(57) ABSTRACT

The invention refers to genetic engineering and can be used in biotechnology, medicine, and agriculture for the manufacture of gene therapy products. Gene therapy DNA vector based on the gene therapy DNA vector VTvaf 17 carrying the therapeutic gene selected from the group of BDNF, VEGFA, BFGF, NGF, GDNF, NT3, CNTF, and IGF1 genes was constructed in order to increase the expression level of this therapeutic gene in humans and animals, while gene therapy DNA vector VTvaf17-BDNF, or VTvaf 17-VEGFA, or VTvaf17-BFGF, or VTvaf17-NGF, or VTvaf17-GDNF, or VTvaf17-NT3, or VTvaf17-CNTF, or VTvaf17-IGFl, has the nucleotide sequence SEQ ID No. 1, or SEQ ID No. 2, or SEQ ID No. 3, or SEQ ID No. 4, or SEQ ID No. 5, or SEQ ID No. 6, or SEQ ID No. 7, or SEQ ID No. 8, respectively.

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and further translation of protein molecules encoded by these genes. Within the description of the invention, gene expression refers to the production of a protein molecule with amino acid sequence encoded by this gene.

BDNF, VEGFA, BFGF, NGF, GDNF, NT3, CNTF, and IGF1 genes included in the group of genes play a key role in several processes in human and animal organisms.

Neurotrophic factors have a proven stimulating effect on the growth of distinct neuronal populations (Aloe et al., 2012, Bothwell, 2014). Delivery of therapeutic substances containing neurotrophic factors to sites with damaged neurons and fibres can be carried out systemically (Sahenk et al., 1994) or locally using osmotic minipumps (Newman et al., 1996b, Utley et al., 1996), slow-release devices (Sterne et al., 1997, Fine et al., 2002, Wood et al., 2009, Wood et al., 2012, Wood et al., 2013) or injections (Chu et al., 2009). Most studies show that important aspects of regeneration, including axon growth, Schwann cell function, and myelination, improve markedly with these approaches (Klimaschewski et al., 2013). At the same time, the response to exogenous neurotrophic factors depends on the nerve type, recovery strategy, and the methodology used to evaluate their effects. Dose, timing, and delivery method are critical parameters that determine efficiency, which indicates, among other things, the existing limitations in the pharmacokinetic parameters of preparations containing the protein substances of these factors. To overcome these limitations, a gene therapy approach can be used.

Viral vectors were successfully used for the expression of neurotrophic factors in Schwann cells to repair damaged nerves in several experimental studies (Dijkhuizen et al., 1998, Hu et al., 2005, Hu et al., 2010, Eggers et al., 2008, Eggers et al., 2013, Tannemaat et al., 2008, Mason et al., 2011). The gene therapy approach was also used to enhance the potential of cell therapy and allogeneic transplants (Shakhbazau et al., 2012, Haastert et al., 2006, Li et al., 2006, Godinho et al., 2013, Santosa et al., 2013). It was also shown that combined gene therapy with the expression of several genes at the same time (BDNF, CNTF, GDNF, NGF, NT3 and VEGF) significantly improved the histological characteristics of tissues, electrophysiological and functional parameters in rats (Hoyng et al., 2014).

The BDNF gene encodes one of the most studied neurotrophic factors in the central nervous system that is involved in the development and maintenance of normal CNS function. It was found that BDNF mediates the survival and differentiation of neurons by binding and activating TrkB receptors localized on both presynaptic and postsynaptic membranes. In addition to the neurotrophic effects, BDNF-TrkB regulates the expression of proteins at different stages of synapse development, and is also involved in brain plasticity. This is particularly important because growing body of evidence demonstrates the important role of BDNF in the pathophysiology of brain-related diseases, including mental disorders. Changes in BDNF expression are widely known in case of depression, schizophrenia, bipolar and anxiety disorders (Polyakova et al., 2015, Mitchelmore et al., 2014, Autry et al., 2012, Briand et al., 2010, Monteleone et al., 2013). Moreover, an increase in the BDNF expression is considered to be one of the potential approaches to the treatment of a number of diseases. Thus, an improvement in cell composition and behavioural tests was shown in a laboratory model of Huntington's disease in rats using an adeno-associated vector expressing this gene (Connor et al., 2016). A similar study demonstrated a neuroprotective action on laboratory mice under oxidative stress (Osborne et al., 2018). Also, systemic administration of cells transfected with a plasmid vector expressing the BDNF gene is proposed as an effective approach to the treatment of rapidly evolving conditions causing CNS damage (e.g. ischemic stroke) (Gomez-Vargas et al., 2012).

The VEGF gene encodes a protein with a well-known angiogenic effect, which is the basis for many studies on its use to stimulate vascular growth in various diseases. However, functions of this growth factor are not limited to this area only. It was shown that VEGF also has direct impact on neurons. Mice with reduced levels of VEGF expression develop degeneration of motor neurons, resembling neurodegenerative disorders in human amyotrophic lateral sclerosis (ALS). Additional genetic studies have confirmed that VEGF is associated with the degeneration of motor neurons in humans and SODI (G93A) mice, i.e. the ALS model. Reduced levels of VEGF expression can contribute to the degeneration of motor neurons by limiting nerve tissue perfusion and VEGF-dependent neuroprotection. VEGF also influences neuronal death after acute ischemia and is involved in other neurological disorders, such as diabetic and ischemic neuropathy, nerve regeneration, Parkinson's disease, Alzheimer's disease, and multiple sclerosis. These data created a base for assessing VEGF potential for the treatment of neurodegenerative disorders. It was shown that intramuscular administration of VEGF-expressing lentiviral vector significantly delayed the onset, improved motor characteristics and enhanced survival of laboratory animals with amyotrophic lateral sclerosis. Data using adeno-associated viral vectors expressing VEGF also showed promising therapeutic effects in ALS (Storkebaum E., Lambrechts D., Carmeliet P.b 2004).

The protein encoded by the BFGF gene (alternative name FGF2 or FGFb) is a member of the fibroblast growth factor (FGF) family. Members of the FGF family of proteins feature a broad spectrum of mitogenic and angiogenic activity. This protein is involved in various biological processes, such as the development of limbs and nervous system, wound healing and tumour growth.

In relation to the processes of neurogenesis, the BFGF injection was highly effective in the regeneration of neurons in several experimental models in laboratory animals, including with regard to damage to the optic nerve (Sapieha P S et al., 2003). Also, several experimental studies have demonstrated the potential of BFGF as a therapeutic agent for neurodegenerative conditions such as Alzheimer's and Parkinson's disease. The adeno-associated viral vector expressing the BFGF gene has the ability to restore spatial learning in mice, hippocampal long-term potentiation, and neurogenesis upon injection both before and after the primary symptoms of Alzheimer's disease. It is important to note that in addition to its neurogenic properties, FGF2 had anti-inflammatory and amyloid-lowering effects (Kiyota T, et al., 2011).

BFGF injection has also been studied as a therapeutic method for the recovery of traumatic brain injury. Rats treated with FGF2 immediately after injury showed enhanced neurogenesis, increase in the number of surviving neurons and improvement in cognitive function compared to control group (Sun D, et al., 2009).

In the experimental model of autoimmune encephalomyelitis in mice, the BFGF neuroprotective role was identified. In one of the studies, intrathecal injection of recombinant herpes simplex virus type 1 (HSV) expressing the human FGF2 gene significantly reduced pathological processes in mice, including, for example, reduction of the number of myelinotoxic cells (T cells and macrophages) in the CNS parenchyma (Ruffini F, et al., 2001).

The NGF gene encodes NGF protein acting as nerve growth factor. NGF is a neutrophin indispensable for the survival and development of sympathetic and sensory neurons. In case of its insufficiency, neurons are susceptible to apoptosis. Nerve growth factor causes axon growth: studies have shown that it contributes to their branching and elongation. NGF prevents or reduces the degeneration of neurons in animals with neurodegenerative diseases. NGF expression is increased in inflammatory diseases in humans in which it suppresses inflammation. In addition, NGF is required for the myelin recovery process. In the study of patients with schizophrenia who have not yet received neuroleptic therapy, it was shown that the NGF level in the cerebrospinal fluid and blood plasma is reduced compared to the normal levels (Kale et al., 2009).

Clinical research on the treatment of Alzheimer's disease is currently under way that involves injection of patients with an adeno-associated vector expressing the NGF gene (NCT00876863). The first results obtained confirm the effectiveness and safety of this approach.

The GDNF gene encodes neurotrophin that contributes to the survival and differentiation of dopaminergic neurons in culture and is able to prevent apoptosis of motor neurons caused by axotomy (Lin et al., 1993). In experiments on rats it was shown that the GDNF injection helps to restore the motor nerve of thigh after traumatic injury (Zhou et al., 2018). Also, through the use of lentiviral vector expressing the GDNF gene, therapeutic effect of the gene therapy approach in the mouse model of Alzheimer's disease was shown (Revilla et al., 2014).

The NT3 gene encodes the neurotrophin protein that ensures the differentiation and survival of existing neurons, and also supports the growth and differentiation of new neurons and synapses. Patients with depression have reduced NT3 concentration in blood serum (Ogłodek et al., 2016). It was also shown that NT3 and BDNF expression is necessary for the recovery of sensory neurons after acoustic trauma (Wan et al., 2014).

NT3 protein has been studied as a treatment for constipation. In a randomised, double-blind, placebo-controlled phase II study, subcutaneous injection of neurotrophin-3 three times a week significantly increased the frequency of spontaneous complete evacuations and increased the effects of other treatments for constipation (Parkman et al., 2003). In various experimental studies on laboratory animals, it was shown that the gene therapy approach using adeno-associated vectors allows an increase in the muscle fibre diameter (Yalvac et al., 2018), reduces inflammation in autoimmune neuropathy (Yalvac et al., 2016), reduces the symptoms of Charcot-Marie-Tooth disease (Sahenk et al., 2014).

The CNTF gene encodes a polypeptide hormone whose action is limited to the nervous system, where it promotes the synthesis of neurotransmitters and regulation of certain populations of neurons. The protein is a powerful survival factor for neurons and oligodendrocytes and may be important for reducing tissue destruction during inflammatory processes, e.g. in sepsis (Guillard et al., 2013). Evidence has shown that CNTF plays an important protective role in retinopathies (Rhee et al., 2013). At the same time, transplantation of cells that overexpress the CNTF gene also has a protective effect in mice with dystrophic retinal changes (Jung et al., 2013).

Mutation in the CNTF gene that leads to aberrant splicing results in a deficiency of the ciliary neurotrophic factor, but the phenotype does not yet have a proven cause-and-effect relationship with any neurological diseases.

The IGF1 gene encodes a protein similar in structure and function to insulin. At the same time, enough evidence has been accumulated that testifies to the fact that the insulin signalling pathway plays an important role in various neurological and neurodegenerative processes (Mishra et al., 2018). It is also shown that IGF1 plays a protective role in the process of reducing cognitive function due to aging (Wennberg et al., 2018). In the ALS mouse model, it was shown that the injection of adeno-associated virus vector expressing the IGF1 gene increased the lifespan of laboratory animals (Hu et al., 2018). Intranasal administration of IGF1 protein was found to reduce electrophysiological phenomena that are manifestations of migraine aura in rats (Grinberg et al., 2017).

Thus, the background of the invention suggests that mutations in BDNF, VEGFA, BFGF, NGF, GDNF, NT3, CNTF, and IGF1 genes or insufficient expression of proteins encoded by these genes are associated with the development of a spectrum of diseases, including, but not limited to, mental and neurodegenerative autoimmune diseases, hereditary and acquired pathological conditions, such as traumatic injuries, and other processes. This is why BDNF, VEGFA, BFGF, NGF, GDNF, NT3, CNTF, and IGF1 genes are grouped within this patent. Genetic constructs that provide expression of proteins encoded by BDNF, VEGFA, BFGF, NGF, GDNF, NT3, CNTF, and IGF1 genes can be used to develop drugs for the prevention and treatment of different diseases and pathological conditions.

Moreover, these data suggest that insufficient expression of proteins encoded by BDNF, VEGFA, BFGF, NGF, GDNF, NT3, CNTF, and IGF1 genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell, and DNA vectors (Guideline on the quality, non-clinical, and clinical aspects of gene therapy medicinal Products EMA/CAT/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N.//Expert Rev Vaccines. 2016; 15 (3): 313-29).

However, limitations of plasmid vectors use in gene therapy are: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products,http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R.//Mol Biotechnol. 2008.39 (2): 97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HELA cells with 383-4548 bp DNA vectors it was shown that the difference in penetration efficiency can be up to two orders of magnitude (100 times different) (Hornstein B D et al.//PLOS ONE. 2016; 11 (12): e0167537.).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2. The plasmid vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

The following applications are prototypes of this invention with regard to the use of gene therapy approaches to increase the expression level of genes from the group of BDNF, VEGFA, BFGF, NGF, GDNF, NT3, CNTF, and IGF1 genes.

Application No. EP0969875A1 describes the invention based on an adenoviral vector expressing NT3 or CNTF gene, and method of usage thereof to protect or repair neurons in diseases or injuries. The disadvantage of this invention is the limitation of genes used and choice of viral vector.

Application No. WO1998056404A1 describes the invention the embodiment of which includes, among others, the use of DNA vectors expressing NGF, or BFGF, or NT3, or BNDF gene to stimulate neuron growth. The disadvantage of this invention is the limitations of gene used and vague efficiency and safety requirements applied to the vectors.

Patent No. U.S. Pat. No. 6,800,281B2 describes invention for the treatment or prevention of neurodegenerative diseases that involves usage of a lentiviral vector expressing the GDNF gene. The disadvantage of this invention is the limitation of genes used and choice of viral vector.

### DISCLOSURE OF THE INVENTION

The purpose of this invention is to construct the gene therapy DNA vectors in order to increase the expression level of a group of BDNF, VEGFA, BFGF, NGF, GDNF, NT3, CNTF, and IGF1 genes in human and animal organisms that combine the following properties:
I) Efficiency of gene therapy DNA vector in order to increase the expression level of therapeutic genes in eukaryotic cells.
II) Possibility of safe use in gene therapy of human beings and animals due to the absence of regulatory elements representing the nucleotide sequences of viral genomes in the gene therapy DNA vector.
III) Possibility of safe use in the gene therapy of human beings and animals due to the absence of antibiotic resistance genes in the gene therapy DNA vector.
IV) Producibility and constructability of gene therapy DNA vector on an industrial scale. Item II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by using the produced gene therapy DNA vector based on the gene therapy DNA vector VTvaf17 for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, such as injuries, neurodegenerative diseases, diabetic neuropathy, conditions resulting in damages to central nervous system, conditions following acute ischemia, for improvement of cognitive functions, as neuroprotective action against oxidative stress, while the gene therapy DNA vector VTvaf17-BDNF contains the coding region of BDNF therapeutic gene cloned to gene therapy DNA vector VTvaf17 with the nucleotide sequence SEQ ID No. 1, the gene therapy DNA vector VTvaf17-VEGFA contains the coding region of VEGFA therapeutic gene cloned to gene therapy DNA vector VTvaf17 with the nucleotide sequence SEQ ID No. 2, the gene therapy DNA vector VTvaf17-BFGF contains the coding region of BFGF therapeutic gene cloned to gene therapy DNA vector VTvaf17 with the nucleotide sequence SEQ ID No. 3, the gene therapy DNA vector VTvaf17-NGF contains the coding region of NGF therapeutic gene cloned to gene therapy DNA vector VTvaf17 with the nucleotide sequence SEQ ID No. 4, the gene therapy DNA vector VTvaf17-GDNF contains the coding region of GDNF therapeutic gene cloned to gene therapy DNA vector VTvaf17 with the nucleotide sequence SEQ ID No. 5, the gene therapy DNA vector VTvaf17-NT3 contains the coding region of NT3 therapeutic gene cloned to gene therapy DNA vector VTvaf17 with the nucleotide sequence SEQ ID No. 6, the gene therapy DNA vector VTvaf17-CNTF contains the coding region of CNTF therapeutic gene cloned to gene therapy DNA vector VTvaf17 with the nucleotide sequence SEQ ID No. 7, the gene therapy DNA vector VTvaf17-IGF1 contains the coding region of IGF1 therapeutic gene cloned to gene therapy DNA vector VTvaf17 with the nucleotide sequence SEQ ID No. 8.

Each of the constructed gene therapy DNA vectors, namely VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 due to the limited size of VTvaf17 vector part not exceeding 3200 bp has the ability to efficiently penetrate into human and animal cells and express the BDNF, or VEGFA, or BFGF, or NGF, or GDNF, or NT3, or CNTF, or IGF1 therapeutic gene cloned to it.

Each of the constructed gene therapy DNA vectors, namely VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvaf17 carrying the BDNF, VEGFA, BFGF, NGF, GDNF, NT3, CNTF, IGF1 therapeutic gene was also developed that involves obtaining each of gene therapy DNA vectors: VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 as follows: the coding region of the BDNF, VEGFA, BFGF, NGF, GDNF, NT3, CNTF, or IGF1 therapeutic gene is cloned to gene therapy DNA vector VTvaf17, and gene therapy DNA vector VTvaf17-BDNF, SEQ ID No. 1, or VTvaf17-VEGFA, SEQ ID No. 2, or VTvaf17-BFGF, SEQ ID No. 3, or VTvaf17-NGF, SEQ ID No. 4, or VTvaf17-GDNF, SEQ ID No. 5, or VTvaf17-NT3, SEQ ID No. 6, or VTvaf17-CNTF, SEQ ID No. 7, or VTvaf17-IGF1, SEQ ID No. 8, respectively, is obtained, while the coding region of the BDNF, or VEGFA, or BFGF, or NGF, or GDNF, or NT3, or CNTF, or IGF1 therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by BamHI and HindIII, or EcoRI and HindIII, or SaII and KpnI restriction sites, while the selection is performed without antibiotics,
at the same time, the following oligonucleotides produced for this purpose are used during gene therapy DNA vector VTvaf17-BDNF, SEQ ID No. 1 production for the reverse transcription reaction and PCR amplification:

BDNF_F
GGATCCGCCACCATGACCATCCTTTTCCTTACTATG,
BDNF_R
AGGGAATTCCTATCTTCCCCTTTTAATGGTC,

and the cleaving of amplification product and cloning of the coding region of BDNF gene to gene therapy DNA vector VTvaf17 is performed by BamHI and EcoRI restriction endonucleases,
at the same time, the following oligonucleotides produced for this purpose are used during gene therapy DNA vector VTvaf17-VEGFA, SEQ ID No. 2 production for the reverse transcription reaction and PCR amplification:

VEGFA_F
GGGGGATCCACCATGACGGACAGACAGACAGACACCGC,
VEGFA_R
TTTGGATCCACCATGAACTTTCTGCTGTCTTGGGTGC,

and the cleaving of amplification product and cloning of the coding region of VEGFA gene to gene therapy DNA vector VTvaf17 is performed by BamHI and HindIII restriction endonucleases,
at the same time, the following oligonucleotides produced for this purpose are used during gene therapy DNA vector VTvaf17-BFGF, SEQ ID No. 3 production for the reverse transcription reaction and PCR amplification:

BFGF_F
GAGGAAGCTTCCACCATGGTGGGTGTGGGGGGTGGAGATG,
BFGF_R
GAGGGAATTCTCAGCTCTTAGCAGACATTGGAAGA,

and the cleaving of amplification product and cloning of the coding region of BFGF gene to gene therapy DNA vector VTvaf17 is performed by HindIII and EcoRI restriction endonucleases,
at the same time, the following oligonucleotides produced for this purpose are used during gene therapy DNA vector VTvaf17-NGF, SEQ ID No. 4 production for the reverse transcription reaction and PCR amplification:

NGF_F
TTTGTCGACCACCATGTCCATGTTGTTCTACACTCTGATC,
NGF_R
AATGGTACCTCAGGCTCTTCTCACAGCCTTCC,

and the cleaving of amplification product and cloning of the coding region of NGF gene to gene therapy DNA vector VTvaf17 is performed by SaII and KpnI restriction endonucleases,
at the same time, the following oligonucleotides produced for this purpose are used during gene therapy DNA vector VTvaf17-GDNF, SEQ ID No. 5 production for the reverse transcription reaction and PCR amplification:

GDNF_F
GGGGGATCCACCATGCAGTCTTTGCCTAACAGCAATGG,
GDNF_R
TTTAAGCTTTCAGATACATCCACACCTTTTAGCG,

and the cleaving of amplification product and cloning of the coding region of GDNF gene to gene therapy DNA vector VTvaf17 is performed by BamHI and HindIII restriction endonucleases,
at the same time, the following oligonucleotides produced for this purpose are used during gene therapy DNA vector VTvaf17-NT3, SEQ ID No. 6 production for the reverse transcription reaction and PCR amplification:

NT3_F
AGGATCCACCATGGTTACTTTTGCCACGATC,
NT3_R
TATAAGCTTTCATGTTCTTCCGATTTTTCTC,

and the cleaving of amplification product and cloning of the coding region of NT3 gene to gene therapy DNA vector VTvaf17 is performed by BamHI and HindIII restriction endonucleases,
at the same time, the following oligonucleotides produced for this purpose are used during gene therapy DNA vector VTvaf17-CNTF, SEQ ID No. 7 production for the reverse transcription reaction and PCR amplification:

CNTF_F
TTTGTCGACCACCATGGCTTTCACAGAGCATTCACC,
CNTF_R
AATGGTACCTACATTTTCTTGTTGTTAGCAATATAATGG,

and the cleaving of amplification product and cloning of the coding region of CNTF gene to gene therapy DNA vector VTvaf17 is performed by SaII and KpnI restriction endonucleases,
at the same time, the following oligonucleotides produced for this purpose are used during gene therapy DNA vector VTvaf17-IGF1, SEQ ID No. 8 production for the reverse transcription reaction and PCR amplification:

IGF1_F
TTTGTCGACCACCATGGGAAAAATCAGCAGTCTTCC,
IGF1_R
AATGGTACCTACTTGCGTTCTTCAAATGTACTTCC,

and the cleaving of amplification product and cloning of the coding region of IGF1 gene to gene therapy DNA vector VTvaf17 is performed by SaII and KpnI restriction endonucleases.

A method of use of the gene therapy DNA vector based on gene therapy DNA vector VTvaf17 carrying BDNF, VEGFA, BFGF, NGF, GDNF, NT3, CNTF, and IGF1 therapeutic gene for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, such as injuries, neurodegenerative diseases, diabetic neuropathy, conditions resulting in damages to central nervous system, conditions following acute ischemia, for improvement of cognitive functions, as neuroprotective action against oxidative stress, was developed that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

A method of production of strain for construction of a gene therapy DNA vector for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, such as injuries, neurodegenerative diseases, diabetic neuropathy, conditions resulting in damages to central nervous system, conditions following acute ischemia, for improvement of cognitive functions, as neuroprotective action against oxidative stress was developed that involves making electrocompetent cells of Escherichia coli strain SCS110-AF and subjecting these cells to electroporation with gene therapy DNA vector VTvaf17-BDNF, or gene therapy DNA vector VTvaf17-VEGFA, or gene therapy DNA vector VTvaf17-BFGF, or gene therapy DNA vector VTvaf17-NGF, or gene therapy DNA vector VTvaf17-GDNF, or gene therapy DNA vector VTvaf17-NT3, or gene therapy DNA vector VTvaf17-CNTF, or gene therapy DNA vector VTvaf17-IGF1. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 is obtained.

Escherichia coli strain SCS110-AF/VTvaf17-BDNF carrying the gene therapy DNA vector VTvaf17-BDNF for production thereof allowing for antibiotic-free selection during gene therapy DNA vector production, or Escherichia coli strain SCS110-AF/VTvaf17-VEGFA carrying the gene therapy DNA vector VTvaf17-VEGFA for production thereof allowing for antibiotic-free selection during gene therapy DNA vector production, or Escherichia coli strain SCS110-AF/VTvaf17-BFGF carrying the gene therapy DNA vector VTvaf17-BFGF for production thereof allowing for antibiotic-free selection during gene therapy DNA vector production, or Escherichia coli strain SCS110-AF/VTvaf17-NGF carrying the gene therapy DNA vector VTvaf17-NGF for production thereof allowing for antibiotic-free selection during gene therapy DNA vector production, or Escherichia coli strain SCS110-AF/VTvaf17-GDNF carrying the gene therapy DNA vector VTvaf17-GDNF for production thereof allowing for antibiotic-free selection during gene therapy DNA vector production, or Escherichia coli strain SCS110-AF/VTvaf17-NT3 carrying the gene therapy DNA vector VTvaf17-NT3 for production thereof allowing for antibiotic-free selection during gene therapy DNA vector production, or Escherichia coli strain SCS110-AF/VTvaf17-CNTF carrying the gene therapy DNA vector VTvaf17-CNTF for production thereof allowing for antibiotic-free selection during gene therapy DNA vector production, or Escherichia coli strain SCS110-AF/VTvaf17-IGF1 carrying the gene therapy DNA vector VTvaf17-IGF1 for production thereof allowing for antibiotic-free selection during gene therapy DNA vector production is claimed for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, such as injuries, neurodegenerative diseases, diabetic neuropathy, conditions resulting in damages to central nervous system, conditions following acute ischemia, for improvement of cognitive functions, as neuroprotective action against oxidative stress.

A method of production on an industrial scale of gene therapy DNA vector based on gene therapy DNA vector VTvaf17 carrying the BDNF, or VEGFA, or BFGF, or NGF, or GDNF, or NT3, or CNTF, or IGF1 therapeutic gene for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, such as injuries, neurodegenerative diseases, diabetic neuropathy, conditions resulting in damages to central nervous system, conditions following acute ischemia, for improvement of cognitive functions, as neuroprotective action against oxidative stress was developed that involves production of gene therapy DNA vector VTvaf17-BDNF, or gene therapy DNA vector VTvaf17-VEGFA, or gene therapy DNA vector VTvaf17-BFGF, or gene therapy DNA vector VTvaf17-NGF, or gene therapy DNA vector VTvaf17-GDNF, or gene therapy DNA vector VTvaf17-NT3, or gene therapy DNA vector VTvaf17-CNTF, or gene therapy DNA vector VTvaf17-IGF1 by inoculating a culture flask containing the prepared medium with seed culture selected from Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1, then the cell culture is incubated in an incubator shaker and transferred to an industrial fermenter, then grown to a stationary phase, then the fraction containing the target DNA product is extracted, multi-stage filtered, and purified by chromatographic methods.

### BRIEF DESCRIPTION OF THE DRAWINGS

The essence of the invention is explained in the drawings, where:

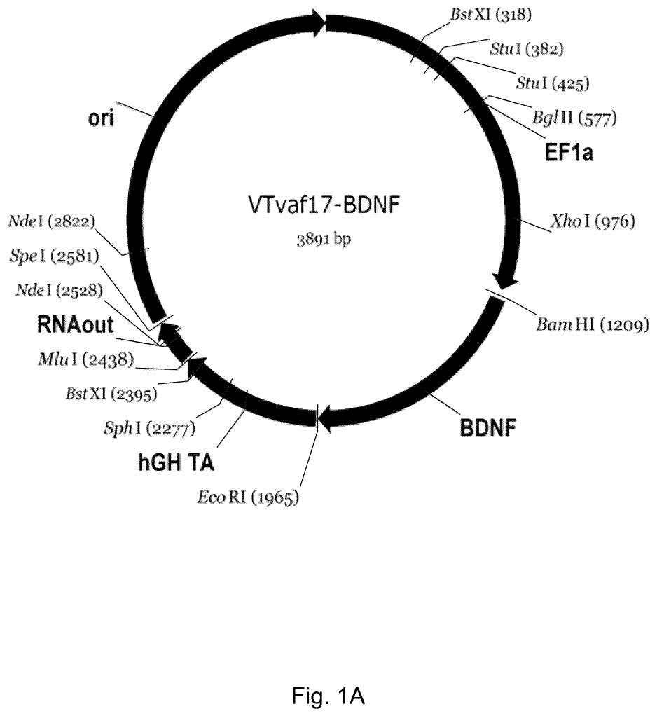

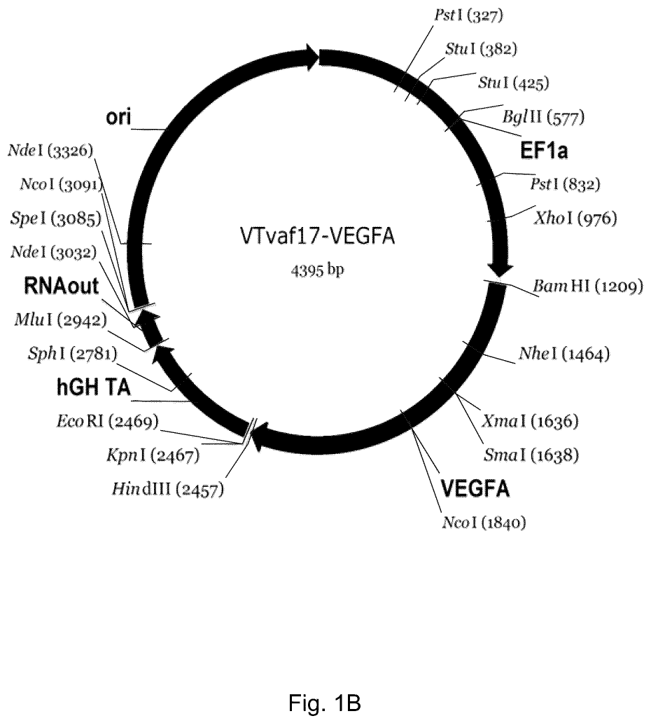

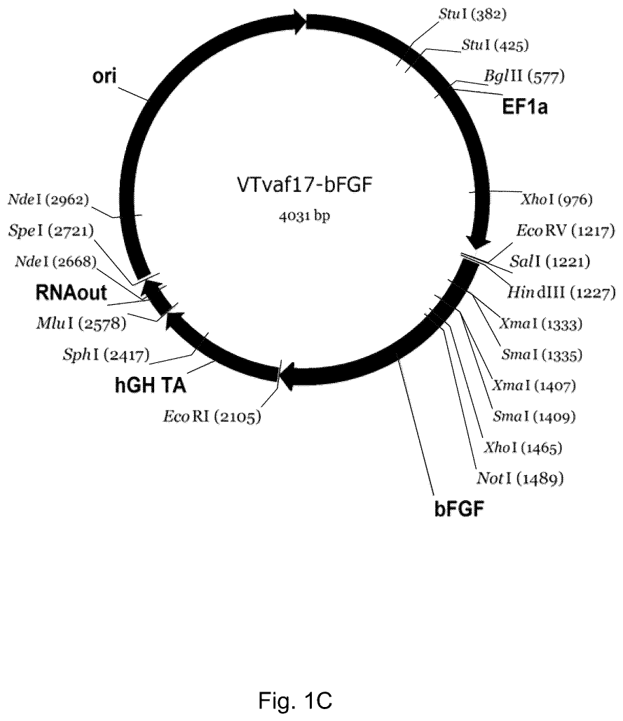

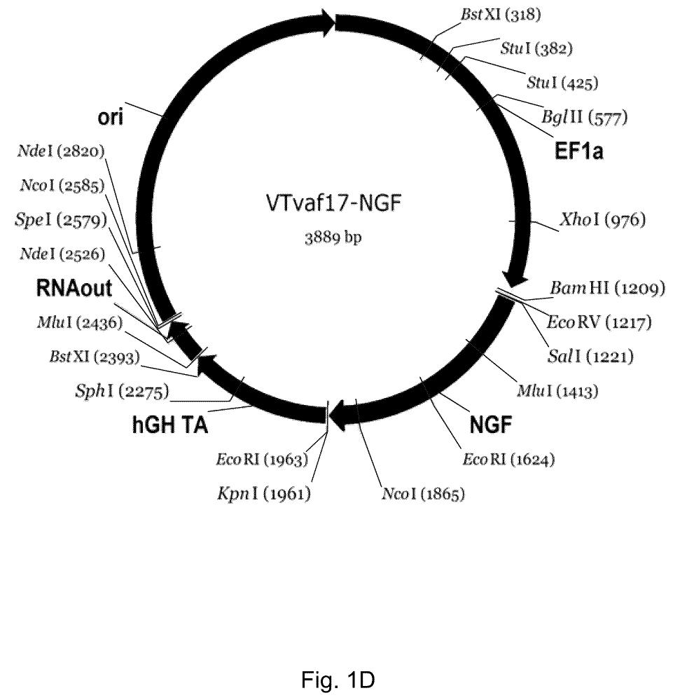

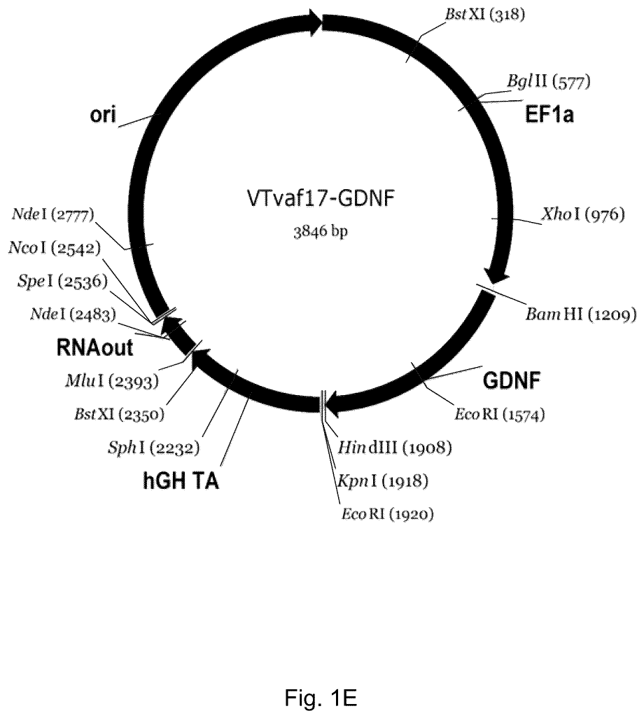

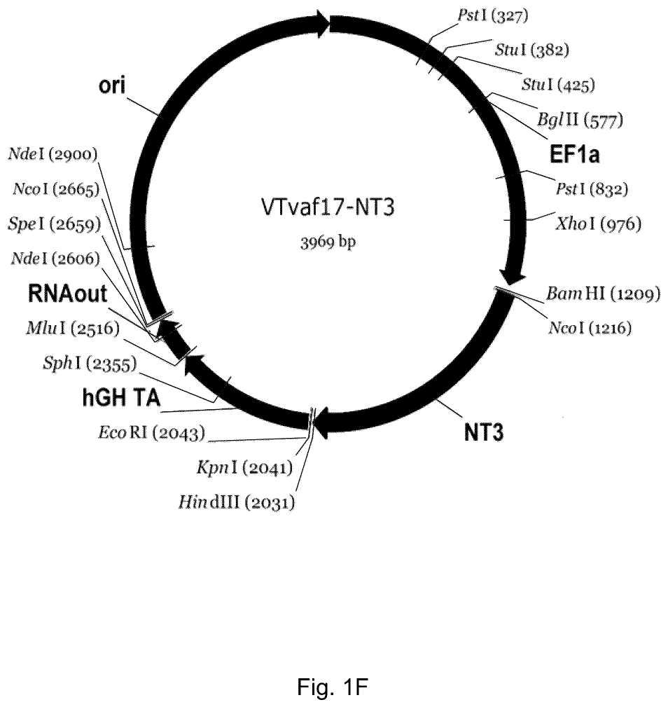

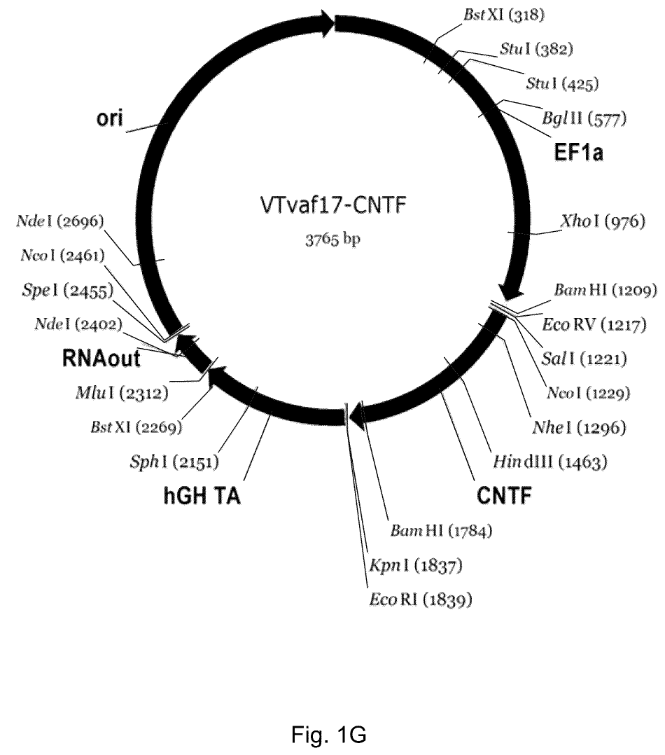

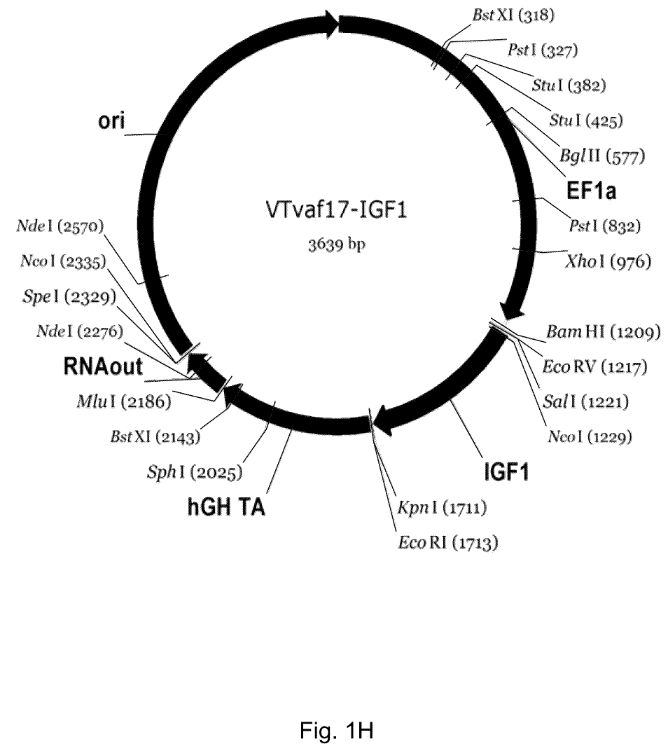

FIG. 1
shows the structure of gene therapy DNA vector VTvaf17 carrying the therapeutic gene selected from the group of BDNF, VEGFA, BFGF, NGF, GDNF, NT3, CNTF, and IGF1 genes that constitutes a circular double-stranded DNA molecule capable of autonomous replication in Escherichia coli cells.

FIG. 1 shows the structures corresponding to:
A-gene therapy DNA vector VTvaf17-BDNF,
B-gene therapy DNA vector VTvaf17-VEGFA,
C-gene therapy DNA vector VTvaf17-BFGF,
D-gene therapy DNA vector VTvaf17-NGF,
E-gene therapy DNA vector VTvaf17-GDNF,
F-gene therapy DNA vector VTvaf17-NT3,
G-gene therapy DNA vector VTvaf17-CNTF,
H-gene therapy DNA vector VTvaf17-IGF1.

The following structural elements of the vector are indicated in the structures:
EF1a—the promoter region of human elongation factor EF1A with an intrinsic enhancer contained in the first intron of the gene. It ensures efficient transcription of the recombinant gene in most human tissues,
The reading frame of the therapeutic gene corresponding to the coding region of the BDNF gene (FIG. 1A), VEGFA gene (FIG. 1B), BFGF gene (FIG. 1C), NGF gene (FIG. 1D), GDNF gene (FIG. 1E), NT3 gene (FIG. 1F), CNTF gene (FIG. 1G), IGF1 gene (FIG. 1H), respectively;
hGH-TA—the transcription terminator and the polyadenylation site of the human growth factor gene,
ori—the origin of replication for autonomous replication with a single nucleotide substitution to increase plasmid production in the cells of most Escherichia coli strains,
RNA-out—the regulatory element RNA-out of transposon Tn 10 allowing for antibiotic-free positive selection in case of the use of Escherichia coli strain SCS 110-AF. Unique restriction sites are marked.

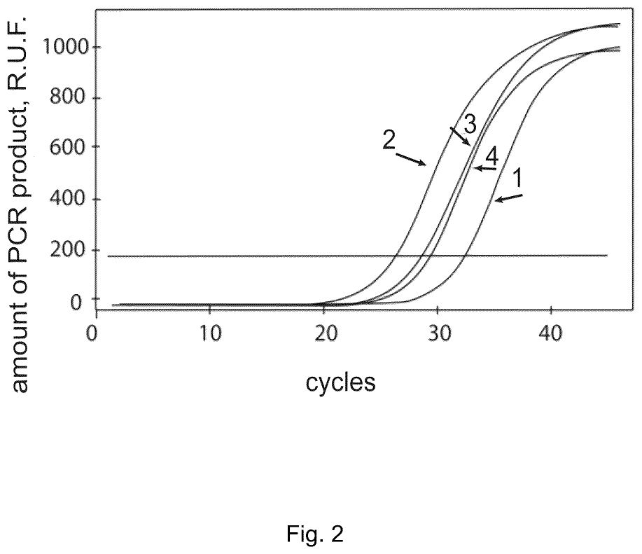

FIG. 2
shows diagrams of cDNA amplicon accumulation of the therapeutic gene, namely the BDNF gene, in human primary skeletal muscle myoblast cells HSKM (Gibco cat #A12555) before their transfection and 48 hours after transfection of these cells with gene therapy DNA vector VTvaf17-BDNF in order to assess the ability to penetrate into eukaryotic cells and functional activity, i.e. expression of the therapeutic gene at the mRNA level.

The following curves of accumulation of amplicons during the reaction are shown in FIG. 2 corresponding to:
1-cDNA of BDNF gene in HSkM primary human skeletal myoblast cell culture before transfection with DNA vector VTvaf17-BDNF,
2-cDNA of BDNF gene in HSkM primary human skeletal myoblast cell culture after transfection with DNA vector VTvaf17-BDNF,
3-cDNA of B2M gene in HSkM primary human skeletal myoblast cell culture before transfection with DNA vector VTvaf17-BDNF,
4-cDNA of B2M gene in HSkM primary human skeletal myoblast cell culture after transfection with DNA vector VTvaf17-BDNF.

B2M (beta-2-microglobuline) gene listed in the GenBank database under number NM 004048.2 was used as a reference gene.

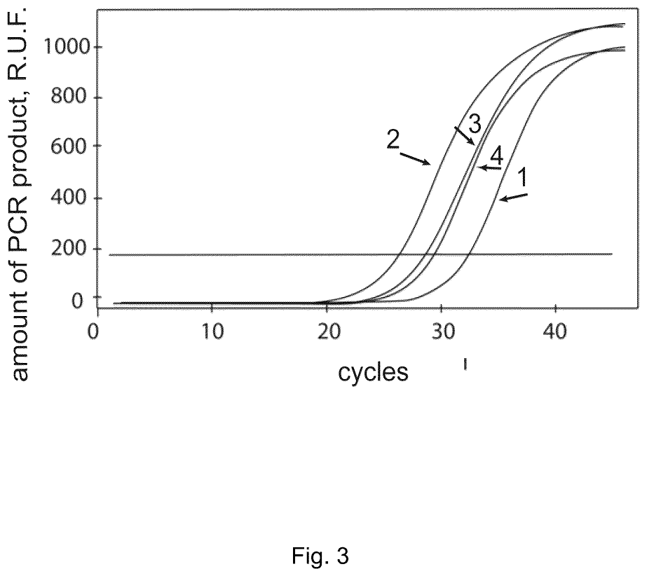

FIG. 3
shows diagrams of cDNA amplicon accumulation of the therapeutic gene, namely the VEGFA gene, in HBdSMC primary human urinary bladder smooth muscle cells culture (ATCC PCS-420-012) before its transfection and 48 hours after transfection of these cells with gene therapy DNA vector VTvaf17-VEGFA in order to assess the ability to penetrate into eukaryotic cells and functional activity, i.e. expression of the therapeutic gene at the mRNA level.

The following curves of accumulation of amplicons during the reaction are shown in FIG. 3 corresponding to:
1-cDNA of VEGFA gene in HBdSMC primary human urinary bladder smooth muscle cells before transfection with DNA vector VTvaf17-VEGFA,
2-cDNA of VEGFA gene in HBdSMC primary human urinary bladder smooth muscle cells after transfection with DNA vector VTvaf17-VEGFA,
3-cDNA of B2M gene in HBdSMC primary human urinary bladder smooth muscle cells before transfection with DNA vector VTvaf17-VEGFA,
4-cDNA of B2M gene in HBdSMC primary human urinary bladder smooth muscle cells after transfection with DNA vector VTvaf17-VEGFA.

B2M (beta-2-microglobuline) gene listed in the GenBank database under number NM 004048.2 was used as a reference gene.

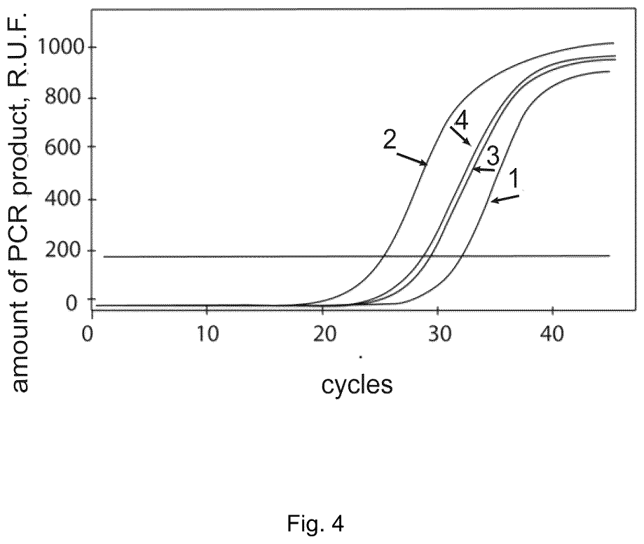

FIG. 4
shows diagrams of cDNA amplicon accumulation of the therapeutic gene, namely the BFGF gene, in T/G HA-VSMC primary human aortic smooth muscle cells (ATCC CRL-1999™) before their transfection and 48 hours after transfection of these cells with gene therapy DNA vector VTvaf17-BFGF in order to assess the ability to penetrate into eukaryotic cells and functional activity, i.e. expression of the therapeutic gene at the mRNA level.

The following curves of accumulation of amplicons during the reaction are shown in FIG. 4 corresponding to:
1-cDNA of BFGF gene in T/G HA-VSMC primary human aortic smooth muscle cells before transfection with DNA vector VTvaf17-BFGF,
2-cDNA of BFGF gene in T/G HA-VSMC primary human aortic smooth muscle cells after transfection with DNA vector VTvaf17-BFGF,
3-cDNA of B2M gene in T/G HA-VSMC primary human aortic smooth muscle cells before transfection with DNA vector VTvaf17-BFGF,
4-cDNA of B2M gene in T/G HA-VSMC primary human aortic smooth muscle cells after transfection with DNA vector VTvaf17-BFGF.

B2M (beta-2-microglobuline) gene listed in the GenBank database under number NM 004048.2 was used as a reference gene.

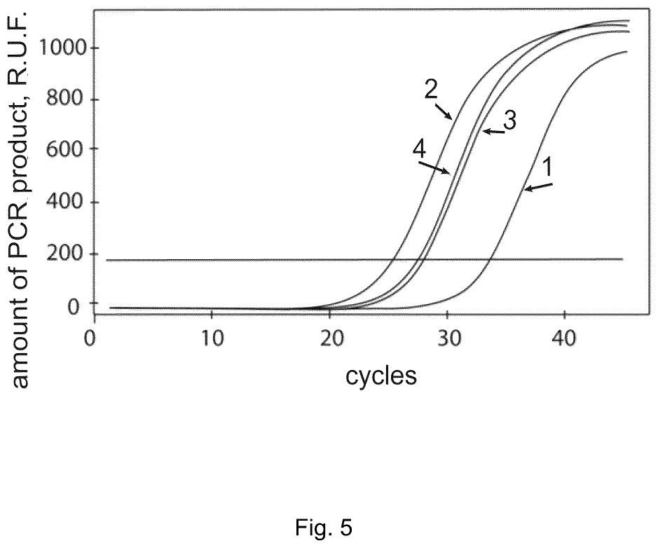

FIG. 5
shows diagrams of cDNA amplicon accumulation of the therapeutic gene, namely the NGF gene, in HUVEC primary umbilical vein endothelial cells (ATCC® PCS-100-013™) before their transfection and 48 hours after transfection of these cells with gene therapy DNA vector VTvaf17-NGF in order to assess the ability to penetrate into eukaryotic cells and functional activity, i.e. expression of the therapeutic gene at the mRNA level.

The following curves of accumulation of amplicons during the reaction are shown in FIG. 5 corresponding to:
1-cDNA of NGF gene in HUVEC primary umbilical vein endothelial cells before transfection with DNA vector VTvaf17-NGF,
2-cDNA of NGF gene in HUVEC primary umbilical vein endothelial cells after transfection with DNA vector VTvaf17-NGF,
3-cDNA of B2M gene in HUVEC primary umbilical vein endothelial cells before transfection with DNA vector VTvaf17-NGF,
4-cDNA of B2M gene in HUVEC primary umbilical vein endothelial cells after transfection with DNA vector VTvaf17-NGF.

B2M (beta-2-microglobuline) gene listed in the GenBank database under number NM 004048.2 was used as a reference gene.

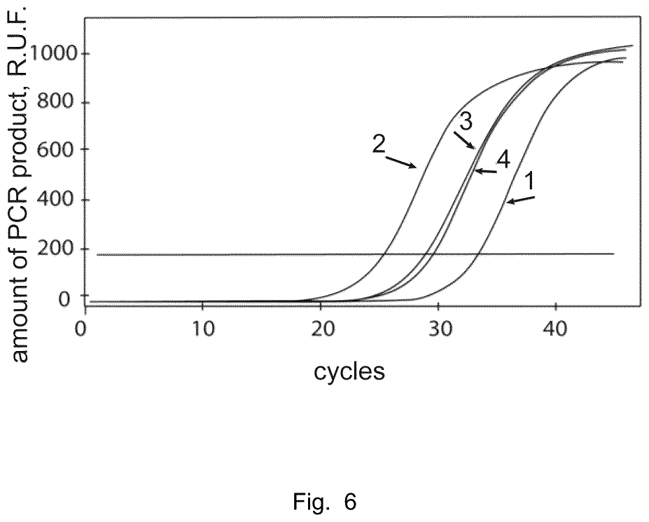

FIG. 6
shows diagrams of cDNA amplicon accumulation of the therapeutic gene, namely the GDNF gene, in HMEC-1 human dermal microvascular endothelial cell culture (ATCC CRL-3243) before its transfection and 48 hours after transfection of these cells with gene therapy DNA vector VTvaf17-GDNF in order to assess the ability to penetrate into eukaryotic cells and functional activity, i.e. expression of the therapeutic gene at the mRNA level.

The following curves of accumulation of amplicons during the reaction are shown in FIG. 6 corresponding to:
1-cDNA of GDNF gene in HMEC-1 human dermal microvascular endothelial cell culture before transfection with DNA vector VTvaf17-GDNF,
2-cDNA of GDNF gene in HMEC-1 human dermal microvascular endothelial cell culture after transfection with DNA vector VTvaf17-GDNF,
3-cDNA of B2M gene in HMEC-1 human dermal microvascular endothelial cell culture before transfection with DNA vector VTvaf17-GDNF,
4-cDNA of B2M gene in HMEC-1 human dermal microvascular endothelial cell culture after transfection with DNA vector VTvaf17-GDNF.

B2M (beta-2-microglobuline) gene listed in the GenBank database under number NM 004048.2 was used as a reference gene.

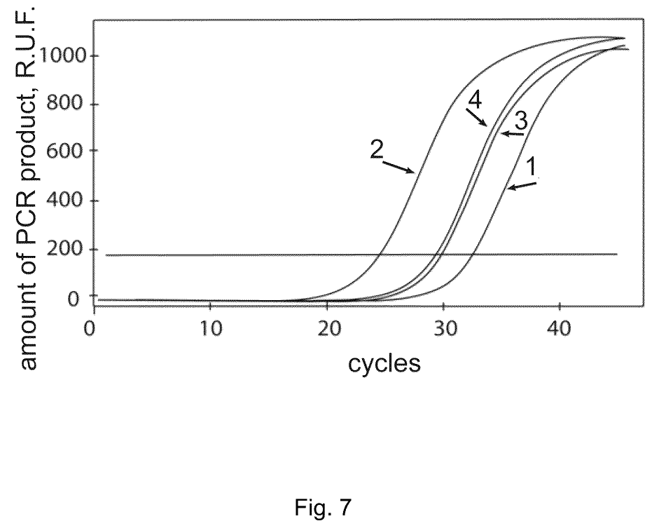

FIG. 7
shows diagrams of cDNA amplicon accumulation of the therapeutic gene, namely the NT3 gene, in SH-SY5Y human neuroblastoma cell culture (ATCC® CRL-2266™) before its transfection and 48 hours after transfection of these cells with gene therapy DNA vector VTvaf17-NT3 in order to assess the ability to penetrate into eukaryotic cells and functional activity, i.e. expression of the therapeutic gene at the mRNA level.

The following curves of accumulation of amplicons during the reaction are shown in FIG. 7 corresponding to:
1-cDNA of NT3 gene in SH-SY5Y human neuroblastoma cell culture before transfection with DNA vector VTvaf17-NT3,
2-cDNA of NT3 gene in SH-SY5Y human neuroblastoma cell culture after transfection with DNA vector VTvaf17-NT3,
3-cDNA of B2M gene in SH-SY5Y human neuroblastoma cell culture before transfection with DNA vector VTvaf17-NT3,
4-cDNA of B2M gene in SH-SY5Y human neuroblastoma cell culture after transfection with DNA vector VTvaf17-NT3.

B2M (beta-2-microglobuline) gene listed in the GenBank database under number NM 004048.2 was used as a reference gene.

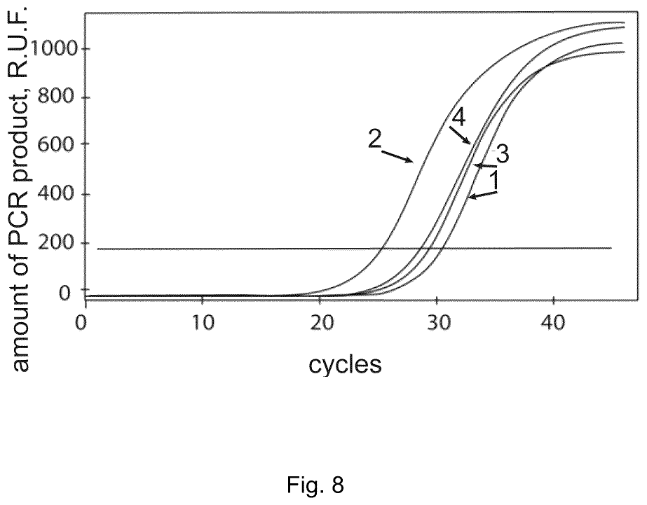

FIG. 8
shows diagrams of cDNA amplicon accumulation of the therapeutic gene, namely the CNTF gene, in primary human corneal epithelial cells (ATCC® PCS-700-010™) before their transfection and 48 hours after transfection of these cells with gene therapy DNA vector VTvaf17-CNTF in order to assess the ability to penetrate into eukaryotic cells and functional activity, i.e. expression of the therapeutic gene at the mRNA level.

The following curves of accumulation of amplicons during the reaction are shown in FIG. 8 corresponding to:
1-cDNA of CNTF gene in primary human corneal epithelial cells before transfection with DNA vector VTvaf17-CNTF,
2-cDNA of CNTF gene in primary human corneal epithelial cells after transfection with DNA vector VTvaf17-CNTF,
3-cDNA of B2M gene in primary human corneal epithelial cells before transfection with DNA vector VTvaf17-CNTF,
4-cDNA of B2M gene in primary human corneal epithelial cells after transfection with DNA vector VTvaf17-CNTF.

B2M (beta-2-microglobuline) gene listed in the GenBank database under number NM 004048.2 was used as a reference gene.

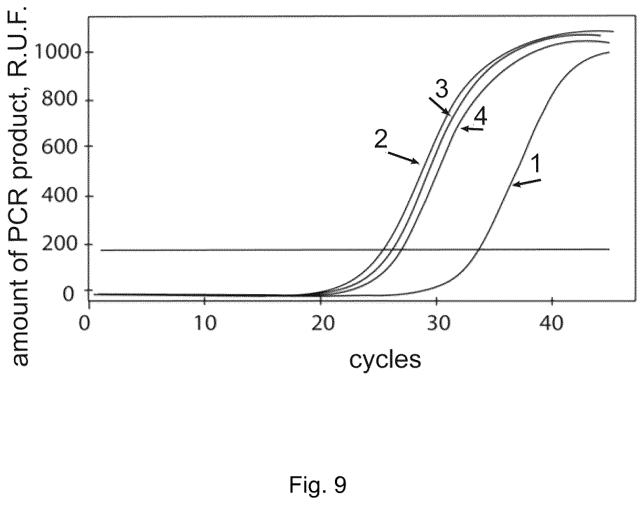

FIG. 9
shows diagrams of cDNA amplicon accumulation of the therapeutic gene, namely the IGF1 gene, in HMEC primary human mammary epithelial cells (ATCC® PCS-600-010™) before their transfection and 48 hours after transfection of these cells with gene therapy DNA vector VTvaf17-IGF1 in order to assess the ability to penetrate into eukaryotic cells and functional activity, i.e. expression of the therapeutic gene at the mRNA level.

The following curves of accumulation of amplicons during the reaction are shown in FIG. 9 corresponding to:
1-cDNA of IGF1 gene in HMEC primary human mammary epithelial cells before transfection with the DNA vector VTvaf17-IGF1,
2-cDNA of IGF1 gene in HMEC primary human mammary epithelial cells after transfection with the DNA vector VTvaf17-IGF1,
3-cDNA of B2M gene in HMEC primary human mammary epithelial cells before transfection with the DNA vector VTvaf17-IGF1,
4-cDNA of B2M gene in HMEC primary human mammary epithelial cells after transfection with the DNA vector VTvaf17-IGF1.

B2M (beta-2-microglobuline) gene listed in the GenBank database under number NM 004048.2 was used as a reference gene.

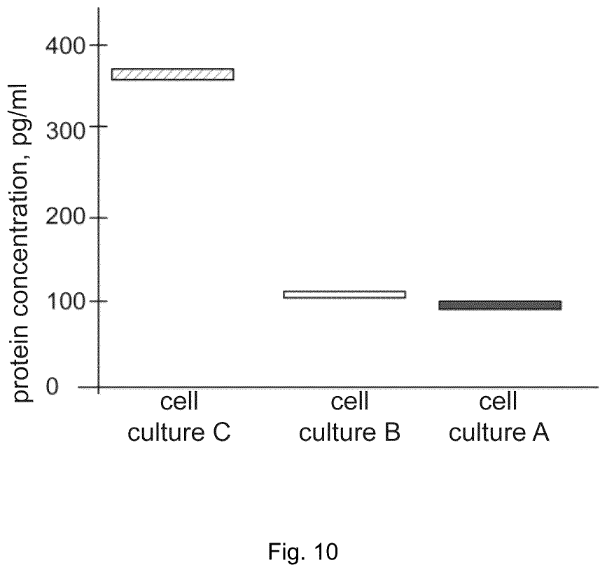

FIG. 10
shows the plot of BDNF protein concentration in the cell lysate of HSkM human primary skeletal muscle myoblast cells (Gibco cat #A12555) after transfection of these cells with DNA vector VTvaf17-BDNF in order to assess the functional activity, i.e. expression at the protein level based on the BDNF protein concentration change in the cell lysate.

The following elements are indicated in FIG. 10:
culture A-HSkM human primary skeletal muscle myoblast cells transfected with aqueous dendrimer solution without plasmid DNA (reference),
culture B-HSKM human primary skeletal muscle myoblast cells transfected with DNA vector VTvaf17,
culture C-HSKM human primary skeletal muscle myoblast cells transfected with DNA vector VTvaf17-BDNF.

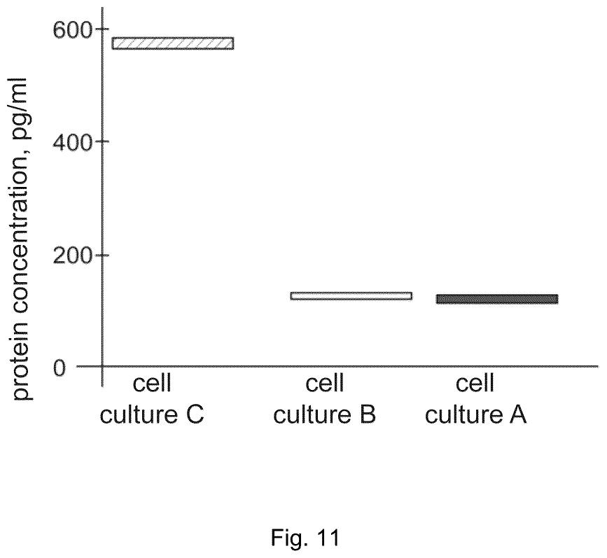

FIG. 11
shows the plot of VEGFA protein concentration in the lysate of HBdSMC human urinary bladder smooth muscle cells (ATCC PCS-420-012) after transfection of these cells with gene therapy DNA vector VTvaf17-VEGFA in order to assess the functional activity, i.e. the therapeutic gene expression at the protein level, and the possibility of increasing the level of protein expression by gene therapy DNA vector based on gene therapy DNA vector VTvaf17 carrying the VEGFA therapeutic gene.

The following elements are indicated in FIG. 11:
culture A-HBdSMC human urinary bladder smooth muscle cells transfected with aqueous dendrimer solution without DNA vector (reference),
culture B-HBdSMC human urinary bladder smooth muscle cells transfected with DNA vector VTvaf17,
culture C-HBdSMC human urinary bladder smooth muscle cells transfected with DNA vector VTvaf17-VEGFA.

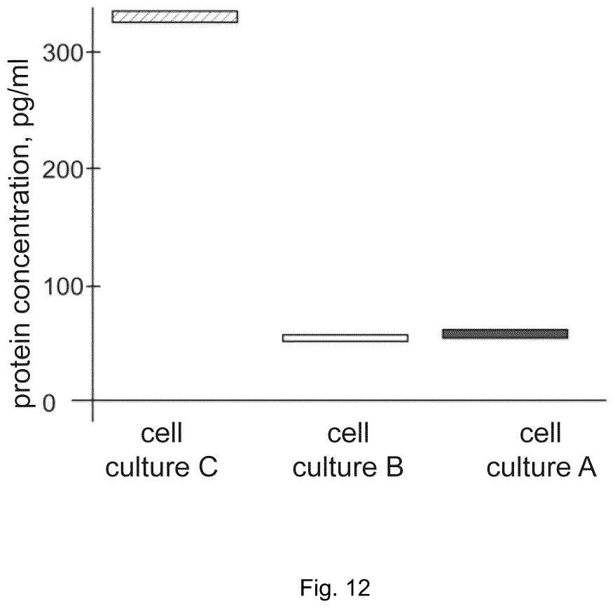

FIG. 12
shows the plot of BFGF protein concentration in the lysate of T/G HA-VSMC primary human aortic smooth muscle cells (ATCC CRL-1999™) after transfection of these cells with gene therapy DNA vector VTvaf17-BFGF in order to assess the functional activity, i.e. the therapeutic gene expression at the protein level, and the possibility of increasing the level of protein expression by gene therapy DNA vector based on gene therapy DNA vector VTvaf17 carrying the BFGF therapeutic gene.

The following elements are indicated in FIG. 12:
culture A-T/G HA-VSMC primary human aortic smooth muscle cells transfected with aqueous dendrimer solution without DNA vector (reference),
culture B-T/G HA-VSMC primary human aortic smooth muscle cells transfected with DNA vector VTvaf17,
culture C-T/G HA-VSMC primary human aortic smooth muscle cells transfected with DNA vector VTvaf17-BFGF.

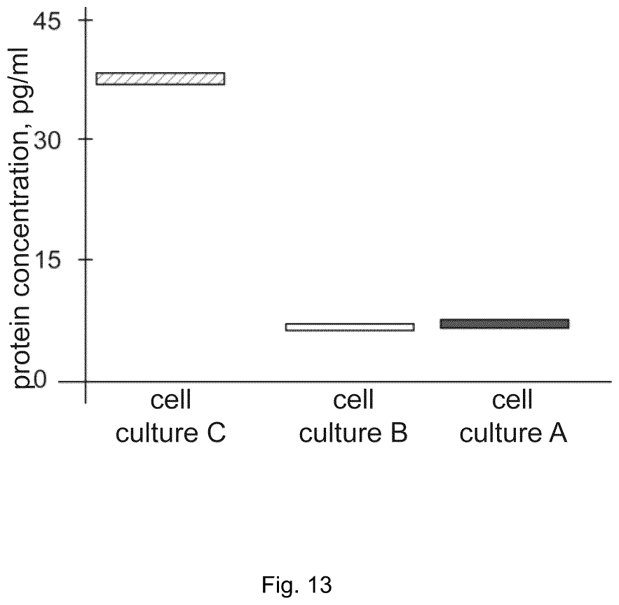

FIG. 13
shows the plot of NGF protein concentration in the lysate of HUVEC primary human umbilical vein endothelial cells (ATCC® PCS-100-013™) after transfection of these cells with DNA vector VTvaf17-NGF in order to assess the functional activity, i.e. the therapeutic gene expression at the protein level, and the possibility of increasing the level of protein expression by gene therapy DNA vector based on gene therapy vector VTvaf17 carrying the NGF therapeutic gene.

The following elements are indicated in FIG. 13:
culture A-HUVEC human umbilical vein endothelial cell culture transfected with aqueous dendrimer solution without plasmid DNA (reference),
culture B-HUVEC human umbilical vein endothelial cell culture transfected with DNA vector VTvaf17,
culture C-HUVEC human umbilical vein endothelial cell culture transfected with DNA vector VTvaf17-NGF.

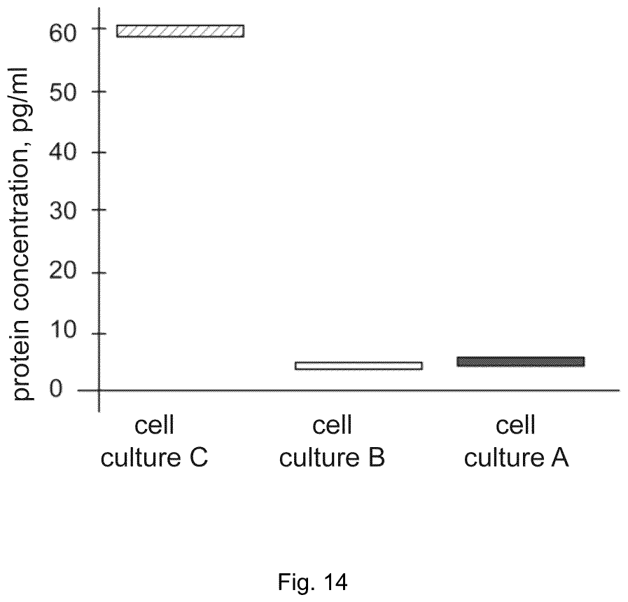

FIG. 14
shows the plot of GDNF protein concentration in the lysate of HMEC-1 human dermal microvascular endothelial cell culture (ATCC CRL-3243) after transfection of these cells with DNA vector VTvaf17-GDNF in order to assess the functional activity, i.e. expression at the protein level based on the GDNF protein concentration change in the cell lysate.

The following elements are indicated in FIG. 14:
culture A-HMEC-1 human dermal microvascular endothelial cell line transfected with aqueous dendrimer solution without plasmid DNA (reference),
culture B-HMEC-1 human dermal microvascular endothelial cell line transfected with DNA vector VTvaf17,
culture C-HMEC-1 human dermal microvascular endothelial cell line transfected with DNA vector VTvaf17-GDNF.

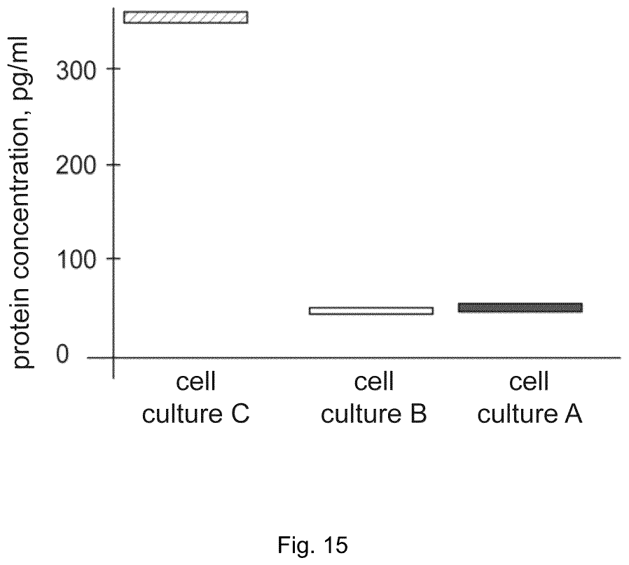

FIG. 15
shows the plot of NT3 protein concentration in the lysate of SH-SY5Y human neuroblastoma cell culture (ATCC® CRL-2266™) after transfection of these cells with DNA vector VTvaf17-NT3 in order to assess the functional activity, i.e. expression at the protein level based on the NT3 protein concentration change in the cell lysate.

The following elements are indicated in FIG. 15:
culture A-SH-SY5Y human neuroblastoma cell culture transfected with aqueous dendrimer solution without plasmid DNA (reference),
culture B—SH-SY5Y human neuroblastoma cell culture transfected with DNA vector VTvaf17,
culture C—SH-SY5Y human neuroblastoma cell culture transfected with DNA vector VTvaf17-NT3.

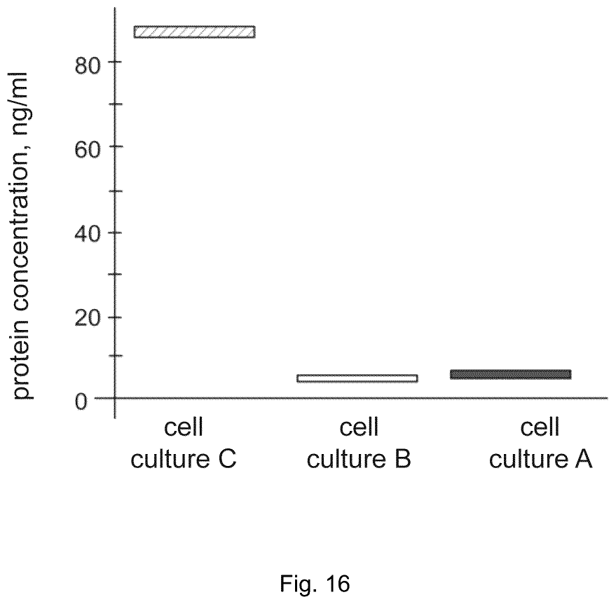

FIG. 16
shows the plot of CNTF protein concentration in the lysate of primary human corneal epithelial cells (ATCC® CRL-700-010™) after transfection of these cells with DNA vector VTvaf17-CNTF in order to assess the functional activity, i.e. expression at the protein level based on the CNTF protein concentration change in the cell lysate.

The following elements are indicated in FIG. 16:
culture A-primary human corneal epithelial cell culture transfected with aqueous dendrimer solution without plasmid DNA (reference),
culture B-primary human corneal epithelial cell culture transfected with DNA vector VTvaf17,
culture C-primary human corneal epithelial cell culture transfected with DNA vector VTvaf17-CNTF.

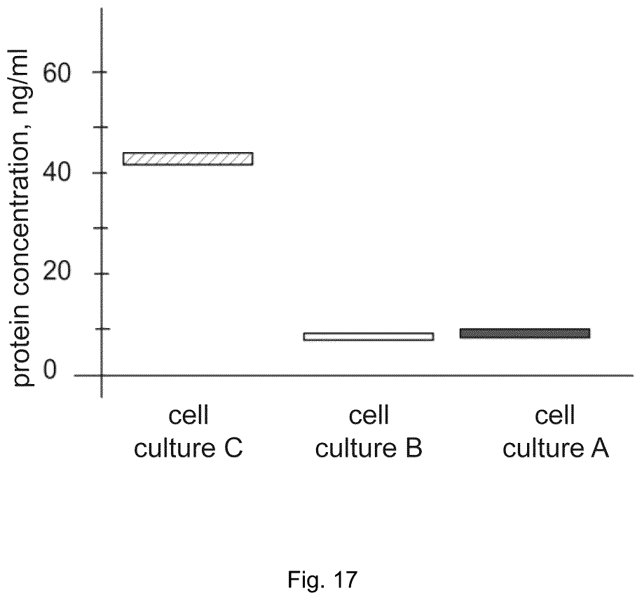

FIG. 17
shows the plot of IGF1 protein concentration in the lysate of primary human mammary epithelial cell culture (ATCC® CRL-600-010™) after transfection of these cells with DNA vector VTvaf17-IGF1 in order to assess the functional activity, i.e. expression at the protein level based on the IGF1 protein concentration change in the cell lysate.

The following elements are indicated in FIG. 17:
culture A-HMEC primary human mammary epithelial cell culture transfected with aqueous dendrimer solution without plasmid DNA (reference),
culture B-HMEC primary human mammary epithelial cell culture transfected with DNA vector VTvaf17,
culture C-HMEC primary human mammary epithelial cell culture transfected with DNA vector VTvaf17-IGF1.

FIG. 18
shows the plot of GDNF protein concentration in the skin biopsy specimens of three patients after injection of gene therapy DNA vector VTvaf17-GDNF into the skin of these patients in order to assess the functional activity, i.e. the expression of the therapeutic gene at the protein level, and the possibility of increasing the level of protein expression using gene therapy DNA vector based on gene therapy vector VTvaf17 carrying the GDNF therapeutic gene.

The following elements are indicated in FIG. 18:
P1I-patient P1 skin biopsy in the region of injection of gene therapy DNA vector VTvaf17-GDNF,
P1II-patient P1 skin biopsy in the region of injection of gene therapy DNA vector VTvaf17 (placebo),
P1III-patient P1 skin biopsy from intact site,
P2I-patient P2 skin biopsy in the region of injection of gene therapy DNA vector VTvaf17-GDNF,
P2II-patient P2 skin biopsy in the region of injection of gene therapy DNA vector VTvaf17 (placebo),
P2III-patient P2 skin biopsy from intact site,
P3I-patient P3 skin biopsy in the region of injection of gene therapy DNA vector VTvaf17-GDNF,
P3II-patient P3 skin biopsy in the region of injection of gene therapy DNA vector VTvaf17 (placebo),
P3III-patient P3 skin biopsy from intact site.

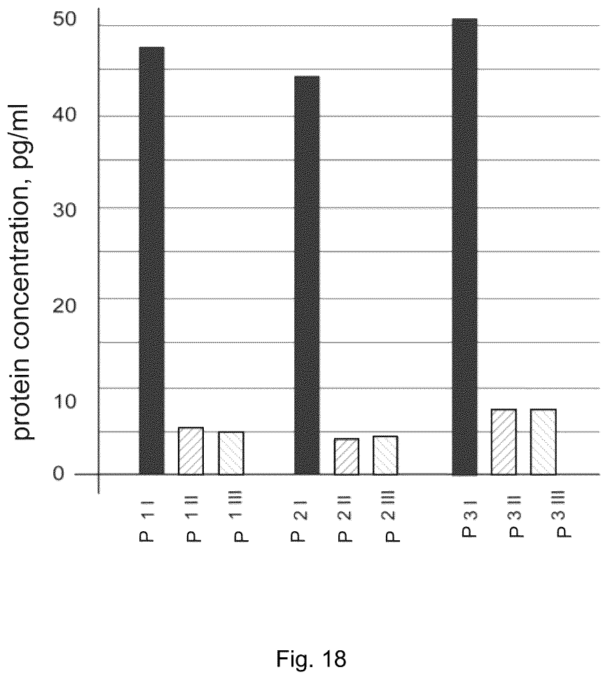

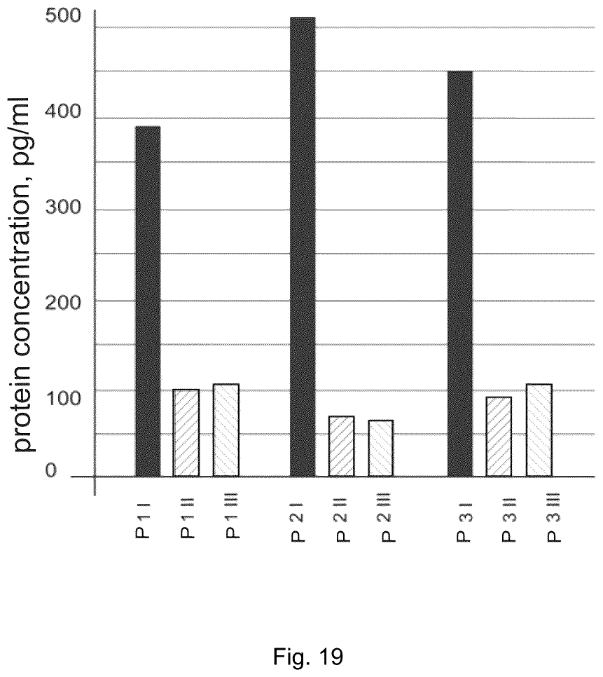

FIG. 19
shows the plot of BDNF protein concentration in the gastrocnemius muscle biopsy specimens of three patients after injection of gene therapy DNA vector VTvaf17-BDNF into the gastrocnemius muscle of these patients in order to assess the functional activity, i.e. the therapeutic gene expression at the protein level, and the possibility of increasing the level of protein expression using gene therapy DNA vector based on gene therapy vector VTvaf17 carrying the BDNF therapeutic gene.

The following elements are indicated in FIG. 19:
P1I-patient P1 gastrocnemius muscle biopsy in the region of injection of gene therapy DNA vector VTvaf17-BDNF,
P1II-patient P1 gastrocnemius muscle biopsy in the region of injection of gene therapy DNA vector VTvaf17 (placebo),
P1III-patient P1 gastrocnemius muscle biopsy from intact site,
P2I-patient P2 gastrocnemius muscle biopsy in the region of injection of gene therapy DNA vector VTvaf17-BDNF,
P2II-patient P2 gastrocnemius muscle biopsy in the region of injection of gene therapy DNA vector VTvaf17 (placebo),
P2III-patient P2 gastrocnemius muscle biopsy from intact site,
P3I-patient P3 gastrocnemius muscle biopsy in the region of injection of gene therapy DNA vector VTvaf17-BDNF,
P3II-patient P3 gastrocnemius muscle biopsy in the region of injection of gene therapy DNA vector VTvaf17 (placebo),
P3III-patient P3 gastrocnemius muscle biopsy from intact site.

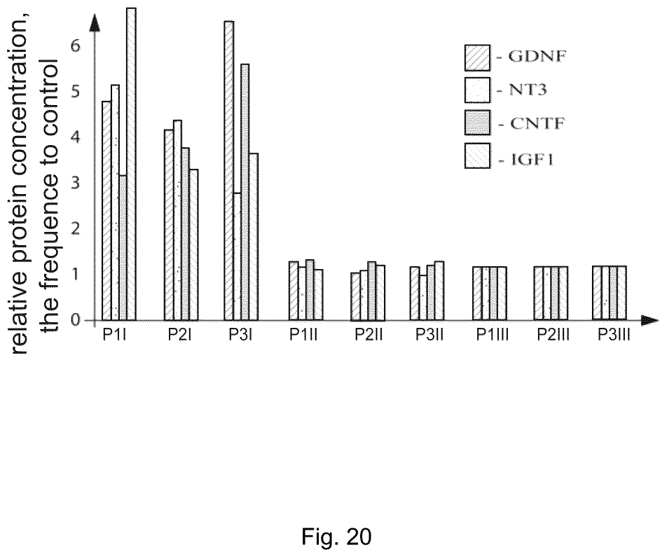

FIG. 20
shows the plot of GDNF, NT3, CNTF, and IGF1 protein concentration in the skin biopsy specimens of three patients after combined injection of gene therapy DNA vector VTvaf17-GDNF, gene therapy DNA vector VTvaf17-NT3, gene therapy DNA vector VTvaf17-CNTF, gene therapy DNA vector VTvaf17-IGF1 into the skin of these patients in order to assess the functional activity, i.e. the expression of the therapeutic gene at the protein level, and the possibility of increasing the level of protein expression using gene therapy DNA vectors based on gene therapy DNA vector VTvaf17 carrying the GDNF, and/or NT3, and/or CNTF, and/or IGF1 therapeutic gene.

The following elements are indicated in FIG. 20:
P1I-patient P1 skin biopsy in the region of injection of a mixture of gene therapy DNA vector VTvaf17-GDNF, gene therapy DNA vector VTvaf17-NT3, gene therapy DNA vector VTvaf17-CNTF, gene therapy DNA vector VTvaf17-IGF1,
P1II-patient P1 skin biopsy in the region of injection of gene therapy DNA vector VTvaf17 (placebo),
P1III-patient P1 skin biopsy from intact site,
P2I-patient P2 skin biopsy in the region of injection of a mixture of gene therapy DNA vector VTvaf17-GDNF, gene therapy DNA vector VTvaf17-NT3, gene therapy DNA vector VTvaf17-CNTF, gene therapy DNA vector VTvaf17-IGF1,
P2II-patient P2 skin biopsy in the region of injection of gene therapy DNA vector VTvaf17 (placebo),
P2III-patient P2 skin biopsy from intact site,
P3I-patient P3 skin biopsy in the region of injection of a mixture of gene therapy DNA vector VTvaf17-GDNF, gene therapy DNA vector VTvaf17-NT3, gene therapy DNA vector VTvaf17-CNTF, gene therapy DNA vector VTvaf17-IGF1,
P3II-patient P3 skin biopsy in the region of injection of gene therapy DNA vector VTvaf17 (placebo),
P3III-patient P3 skin biopsy from intact site.

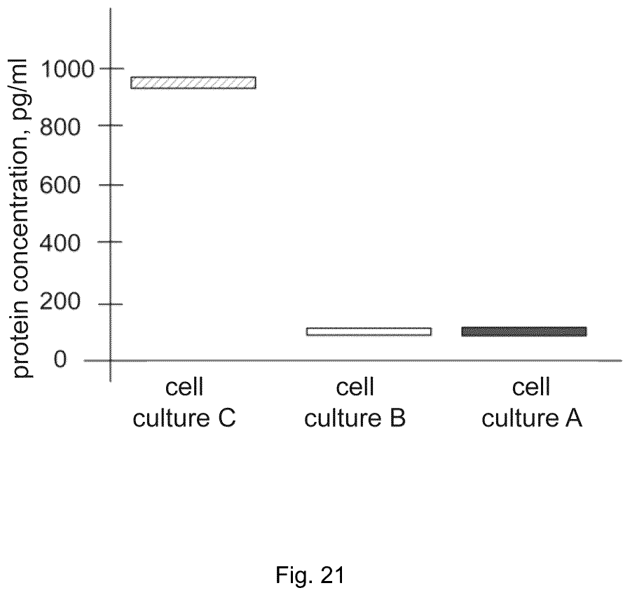

FIG. 21
shows the plot of VEGFA protein concentration in human skin biopsy samples after subcutaneous injection of autologous fibroblast cell culture transfected with the gene therapy DNA vector VTvaf17-VEGFA in order to demonstrate the method of use by injecting autologous cells transfected with the gene therapy DNA vector VTvaf17-VEGFA.

The following elements are indicated in FIG. 21:
P1C-patient P1 skin biopsy in the region of injection of autologous fibroblast culture of the patient transfected with gene therapy DNA vector VTvaf17-VEGFA,
P1B-patient P1 skin biopsy in the region of injection of autologous fibroblasts of the patient transfected with gene therapy DNA vector VTvaf17,
P1A-patient P1 skin biopsy from intact site.

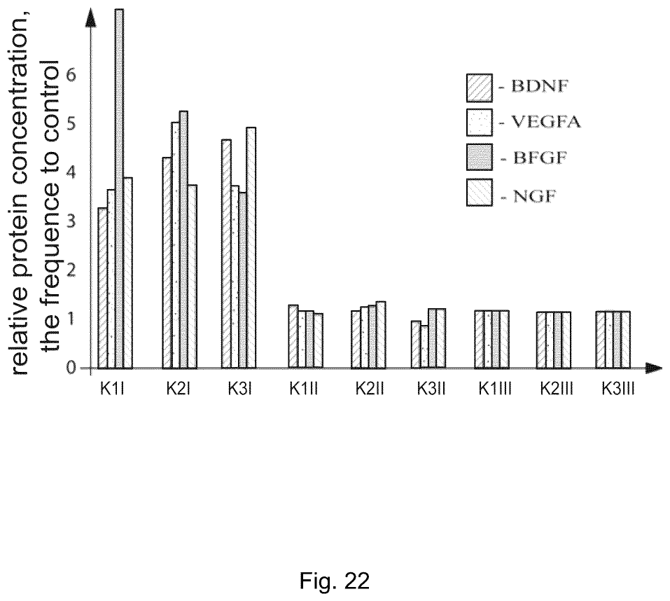

FIG. 22
shows the plot of BDNF, VEGFA, BFGF, and NGF protein concentration in the tibial muscle biopsy samples of three rats after the combined injection of the tibial muscle of these animals with the following gene therapy DNA vectors: VTvaf17-BDNF, VTvaf17-VEGFA, VTvaf17-BFGF, and VTvaf17-NGF in order to assess their functional activity, i.e. the therapeutic gene expression at the protein level, and the possibility of increasing the level of protein expression by gene therapy DNA vectors based on gene therapy vector VTvaf17 carrying the BDNF, and/or VEGFA, and/or BFGF, and/or NGF therapeutic gene.

The following elements are indicated in FIG. 22:
K1I-rat K1 tibial muscle biopsy sample in the region of injection of a mixture of gene therapy DNA vectors: VTvaf17-BDNF, VTvaf17-VEGFA, VTvaf17-BFGF, and VTvaf17-NGF,
K1II-rat K1 tibial muscle biopsy sample in the region of injection of gene therapy DNA vector VTvaf17 (placebo),
K1III-rat K1 tibial muscle biopsy sample of the reference intact site,
K2I-rat K2 tibial muscle biopsy sample in the region of injection of a mixture of gene therapy DNA vectors: VTvaf17-BDNF, VTvaf17-VEGFA, VTvaf17-BFGF, and VTvaf17-NGF,
K2II-rat K2 tibial muscle biopsy sample in the region of injection of gene therapy DNA vector VTvaf17 (placebo),
K2III-rat K2 tibial muscle biopsy sample of the reference intact site,
K31-rat K3 tibial muscle biopsy sample in the region of injection of a mixture of gene therapy DNA vectors: VTvaf17-BDNF, VTvaf17-VEGFA, VTvaf17-BFGF, and VTvaf17-NGF,
K3II-rat K3 tibial muscle biopsy sample in the region of injection of gene therapy DNA vector VTvaf17 (placebo),
K3III-rat K3 tibial muscle biopsy sample of the reference intact site.

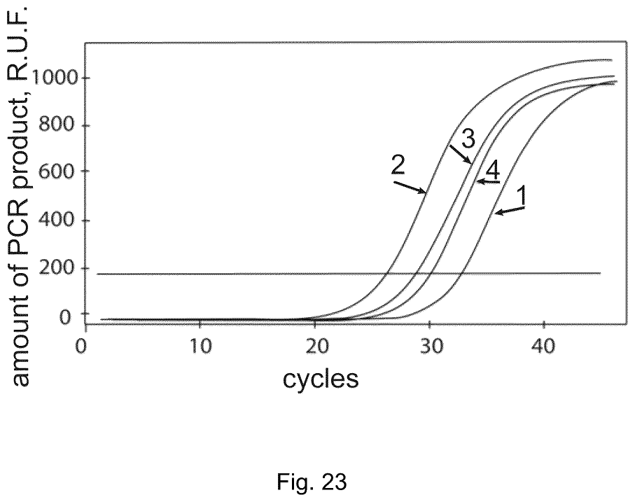

FIG. 23
shows diagrams of cDNA amplicon accumulation of the BFGF therapeutic gene in BAOSMC bovine aortic smooth muscle cells (Genlantis) before and 48 hours after transfection of these cells with the DNA vector VTvaf17-BFGF in order to demonstrate the method of use by injecting the gene therapy DNA vector in animals.

The following curves of accumulation of amplicons during the reaction are shown in FIG. 23 corresponding to:
1-cDNA of BFGF gene in BAOSMC bovine aortic smooth muscle cells before transfection with gene therapy DNA vector VTvaf17-BFGF,
2-cDNA of BFGF gene in BAOSMC bovine aortic smooth muscle cells before transfection with gene therapy DNA vector VTvaf17-BFGF,
3-cDNA of ACT gene in BAOSMC bovine aortic smooth muscle cells before transfection with gene therapy DNA vector VTvaf17-BFGF,
4-cDNA of ACT gene in BAOSMC bovine aortic smooth muscle cells BAOSMC after transfection with gene therapy DNA vector VTvaf17-BFGF.

Bull/cow actin gene (ACT) listed in the GenBank database under number AH001130.2 was used as a reference gene.

### EMBODIMENT OF THE INVENTION

Gene therapy DNA vectors carrying the human therapeutic genes designed to increase the expression level of these therapeutic genes in human and animal tissues were constructed based on 3165 bp DNA vector VTvaf17. The method of production of each gene therapy DNA vector carrying the therapeutic genes is to clone the protein coding sequence of the therapeutic gene selected from the group of the following genes: human BDNF gene (encodes BDNF protein), human VEGFA gene (encodes VEGFA protein), human BFGF gene (encodes BFGF protein), human NGF gene (encodes NGF protein), human GDNF gene (encodes GDNF protein), human NT3 gene (encodes NT3 protein), human CNTF gene (encodes CNTF protein), human IGF1 gene (encodes IGF1 protein) to the polylinker of gene therapy DNA vector VTvaf17. It is known that the ability of DNA vectors to penetrate into eukaryotic cells is due mainly to the vector size. DNA vectors with the smallest size have higher penetration capability. Thus, the absence of elements in the vector that bear no functional load, but at the same time increase the vector DNA size is preferred. These features of DNA vectors were taken into account during the production of gene therapy DNA vectors based on gene therapy DNA vector VTvaf17 carrying the therapeutic gene selected from the group of BDNF, VEGFA, BFGF, NGF, GDNF, NT3, CNTF, and IGF1 genes with no large non-functional sequences and antibiotic resistance genes in the vector, which, in addition to technological advantages and safe use, allowed for the significant reduction of size of the produced gene therapy DNA vector VTvaf17 carrying the therapeutic gene selected from the group of BDNF, VEGFA, BFGF, NGF, GDNF, NT3, CNTF, and IGF1 genes. Thus, the ability of the obtained gene therapy DNA vector to penetrate into eukaryotic cells is due to its small length.

Each of the following gene therapy DNA vectors: VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 was produced as follows: the coding region of the therapeutic gene BDNF, or VEGFA, or BFGF, or NGF, or GDNF, or NT3, or CNTF, or IGF1 was cloned to DNA vector VTvaf17 and gene therapy DNA vector VTvaf17-BDNF, SEQ ID No. 1, or VTvaf17-VEGFA, SEQ ID No. 2, or VTvaf17-BFGF, SEQ ID No. 3, or VTvaf17-NGF, SEQ ID No. 4, or VTvaf17-GDNF, SEQ ID No. 5, or VTvaf17-NT3, SEQ ID No. 6, or VTvaf17-CNTF, SEQ ID No. 7, or VTvaf17-IGF1, SEQ ID No. 8, respectively, was obtained. The coding region of BDNF gene (750 bp), or VEGFA gene (1242 bp), or BFGF gene (872 bp), or NGF gene (726 bp), or GDNF gene (693 bp), or NT3 gene (816 bp), or CNTF gene (607 bp), or IGF1 gene (481 bp) was produced by extracting total RNA from the biological normal tissue sample. The reverse transcription reaction was used for the synthesis of the first chain cDNA of human BDNF, VEGFA, BFGF, NGF, GDNF, NT3, CNTF, and IGF1 genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector VTvaf17 was performed by BamHI and EcoRI, or SaII and KpnI, or BamHI and HindIII restriction sites located in the VTvaf17 vector polylinker. The selection of restriction sites was carried out in such a way that the cloned fragment entered the reading frame of expression cassette of the vector VTvaf17, while the protein coding sequence did not contain restriction sites for the selected endonucleases. Experts in this field realise that the methodological implementation of gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 production can vary within the framework of the selection of known methods of molecular gene cloning and these methods are included in the scope of this invention. For example, different oligonucleotide sequences can be used to amplify BDNF, or VEGFA, or BFGF, or NGF, or GDNF, or NT3, or CNTF, or IGF1 gene, different restriction endonucleases or laboratory techniques, such as ligation independent cloning of genes.

Gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 has the nucleotide sequence SEQ ID No. 1, or SEQ ID No. 2, or SEQ ID No. 3, or SEQ ID No. 4, or SEQ ID No. 5, SEQ ID No. 6, or SEQ ID No. 7, or SEQ ID No. 8, respectively. At the same time, degeneracy of genetic code is known to the experts in this field and means that the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of VTvaf17 vector. At the same time, genetic polymorphism is known to the experts in this field and means that the scope of this invention also includes variants of nucleotide sequences of genes from the group of BDNF, VEGFA, BFGF, NGF, GDNF, NT3, CNTF, and IGF1 genes that also encode different variants of the amino acid sequences of BDNF, VEGFA, BFGF, NGF, GDNF, NT3, CNTF, and IGF1 proteins that do not differ from those listed in their functional activity under physiological conditions.

The ability to penetrate into eukaryotic cells and express functional activity, i.e. the ability to express the therapeutic gene of the obtained gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 is confirmed by injecting the obtained vector into eukaryotic cells and subsequent analysis of the expression of specific mRNA and/or protein product of the therapeutic gene. The presence of specific mRNA in cells into which the gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 was injected shows the ability of the obtained vector to both penetrate into eukaryotic cells and express mRNA of the therapeutic gene. Furthermore, it is known to the experts in this field that the presence of mRNA gene is a mandatory condition, but not an evidence of the translation of protein encoded by the therapeutic gene. Therefore, in order to confirm properties of the gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 to express the therapeutic gene at the protein level in eukaryotic cells into which the gene therapy DNA vector was injected, analysis of the concentration of proteins encoded by the therapeutic genes was carried out using immunological methods. The presence of BDNF, or VEGFA, or BFGF, or NGF, or GDNF, or NT3, or CNTF, or IGF1 protein confirms the efficiency of expression of therapeutic genes in eukaryotic cells and the possibility of increasing the protein concentration using the gene therapy DNA vector based on gene therapy DNA vector VTvaf17 carrying the therapeutic gene selected from the group of BDNF, VEGFA, BFGF, NGF, GDNF, NT3, CNTF, or IGF1 genes.

Thus, in order to confirm the expression efficiency of gene therapy DNA vector VTvaf17-BDNF carrying the therapeutic gene, namely the BDNF gene, gene therapy DNA vector VTvaf17-VEGFA carrying the therapeutic gene, namely the VEGFA gene, gene therapy DNA vector VTvaf17-BFGF carrying the therapeutic gene, namely the BFGF gene, gene therapy DNA vector VTvaf17-NGF carrying the therapeutic gene, namely the NGF gene, gene therapy DNA vector VTvaf17-GDNF carrying the therapeutic gene, namely the GDNF gene, gene therapy DNA vector VTvaf17-NT3 carrying the therapeutic gene, namely the NT3 gene, gene therapy DNA vector VTvaf17-CNTF carrying the therapeutic gene, namely the CNTF gene, gene therapy DNA vector VTvaf17-IGF1 carrying the therapeutic gene, namely the IGF1 gene, the following methods were used:
A) real-time PCR, i.e. change in mRNA accumulation of therapeutic genes in human and animal cell lysate after transfection of different human and animal cell lines with gene therapy DNA vectors,
B) Enzyme-linked immunosorbent assay, i.e. change in the quantitative level of therapeutic proteins in the human cell lysate after transfection of different human cell lines with gene therapy DNA vectors,
C) Enzyme-linked immunosorbent assay, i.e. change in the quantitative level of therapeutic proteins in the supernatant of human and animals tissue biopsy specimens after the injection of gene therapy DNA vectors into these tissues,
D) Enzyme-linked immunosorbent assay, i.e. change in the quantitative level of therapeutic proteins in the supernatant of human tissue biopsies after the injection of these tissues with autologous cells of this human transfected with gene therapy DNA vectors.

In order to confirm the practicability of use of the constructed gene therapy DNA vector VTvaf17-BDNF carrying the therapeutic gene, namely the BDNF gene, gene therapy DNA vector VTvaf17-VEGFA carrying the therapeutic gene, namely the VEGFA gene, gene therapy DNA vector VTvaf17-BFGF carrying the therapeutic gene, namely the BFGF gene, gene therapy DNA vector VTvaf17-NGF carrying the therapeutic gene, namely the NGF gene, gene therapy DNA vector VTvaf17-GDNF carrying the therapeutic gene, namely the GDNF gene, gene therapy DNA vector VTvaf17-NT3 carrying the therapeutic gene, namely the NT3 gene, gene therapy DNA vector VTvaf17-CNTF carrying the therapeutic gene, namely the CNTF gene, gene therapy DNA vector VTvaf17-IGF1 carrying the therapeutic gene, namely the IGF1 gene, the following was performed:
A) transfection of different human and animal cell lines with gene therapy DNA vectors,
B) injection of gene therapy DNA vectors into different human and animal tissues,
C) injection of a mixture of gene therapy DNA vectors into human and animal tissues,
D) injection of autologous cells transfected with gene therapy DNA vectors into human tissues.

These methods of use lack potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of gene therapy DNA vector VTvaf17-BDNF, or gene therapy DNA vector VTvaf17-VEGFA, or gene therapy DNA vector VTvaf17-BFGF, or gene therapy DNA vector VTvaf17-NGF, or gene therapy DNA vector VTvaf17-GDNF, or gene therapy DNA vector VTvaf17-NT3, or gene therapy DNA vector VTvaf17-CNTF, or gene therapy DNA vector VTvaf17-IGF1 (SEQ ID No. 1, or SEQ ID No. 2, or SEQ ID No. 3, or SEQ ID No. 4, or SEQ ID No. 5, or SEQ ID No. 6, or SEQ ID No. 7, or SEQ ID No. 8, respectively).

It is known to the experts in this field that antibiotic resistance genes in the gene therapy DNA vectors are used to obtain these vectors in preparative quantities by increasing bacterial biomass in a nutrient medium containing a selective antibiotic. Within the framework of this invention, in order to ensure the safe use of gene therapy DNA vector VTvaf17 carrying BDNF, or VEGFA, or BFGF, or NGF, or GDNF, or NT3, or CNTF, or IGF1 therapeutic gene, the use of selective nutrient media containing an antibiotic is not possible. A method for obtaining strains for production of these gene therapy vectors based on Escherichia coli strain SCS110-AF is proposed as a technological solution for obtaining the gene therapy DNA vector VTvaf17 carrying a therapeutic gene selected from the group of BDNF, VEGFA, BFGF, NGF, GDNF, NT3, CNTF, and IGF1 genes in order to scale up the production of gene therapy vectors on an industrial scale. The method of Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli SCS110-AF/VTvaf17-CNTF, or Escherichia coli strain SCS110-AF/VTvaf17-IGF1 production involves production of competent cells of Escherichia coli strain SCS110-AF with the injection of gene therapy DNA vector VTvaf17-BDNF, or DNA vector VTvaf17-VEGFA, or DNA vector VTvaf17-BFGF, or DNA vector VTvaf17-NGF, or DNA vector VTvaf17-GDNF, or DNA vector VTvaf17-NT3, or DNA vector VTvaf17-CNTF, or DNA vector VTvaf17-IGF1 into these cells, respectively, using transformation (electroporation) methods widely known to experts in this field. The obtained Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli SCS110-AF/VTvaf17-CNTF, or Escherichia coli strain SCS110-AF/VTvaf17-IGF1 is used to produce the gene therapy DNA vector VTvaf17-BDNF, or DNA vector VTvaf17-VEGFA, or DNA vector VTvaf17-BFGF, or DNA vector VTvaf17-NGF, or DNA vector VTvaf17-GDNF, or DNA vector VTvaf17-NT3, or DNA vector VTvaf17-CNTF, or DNA vector VTvaf17-IGF1, respectively, allowing for the use of antibiotic-free media.

In order to confirm the construction of Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3 or Escherichia coli SCS110-AF/VTvaf17-CNTF, or Escherichia coli strain SCS110-AF/VTvaf17-IGF1, transformation, selection, and subsequent biomass growth with extraction of plasmid DNA were performed.

To confirm the producibility, constructability and scale up of the production of gene therapy DNA vector VTvaf17-BDNF carrying the therapeutic gene, namely BDNF gene, or VTvaf17-VEGFA carrying the therapeutic gene, namely VEGFA gene, or VTvaf17-BFGF carrying the therapeutic gene, namely BFGF gene, or VTvaf17-NGF carrying the therapeutic gene, namely NGF gene, or VTvaf17-GDNF carrying the therapeutic gene, namely GDNF gene, or VTvaf17-NT3 carrying the therapeutic gene, namely NT3 gene, or VTvaf17-CNTF carrying the therapeutic gene, namely CNTF gene, or VTvaf17-IGF1 carrying the therapeutic gene, namely IGF1 gene, to an industrial scale, the fermentation on an industrial scale of Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 each containing gene therapy DNA vector VTvaf17 carrying the therapeutic gene, namely BDNF, or VEGFA, or BFGF, or NGF, or GDNF, or NT3, or CNTF, or IGF1 gene was performed.

The method of scaling the production of bacterial mass to an industrial scale for the isolation of gene therapy DNA vector VTvaf17 carrying the therapeutic gene selected from the group of BDNF, VEGFA, BFGF, NGF, GDNF, NT3, CNTF, and IGF1 genes involves incubation of the seed culture of Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. the gene therapy DNA vector VTvaf17-BDNF, or gene therapy DNA vector VTvaf17-VEGFA, or gene therapy DNA vector VTvaf17-BFGF, or gene therapy DNA vector VTvaf17-NGF, or gene therapy DNA vector VTvaf17-GDNF, or gene therapy DNA vector VTvaf17-NT3, or gene therapy DNA vector VTvaf17-CNTF, or gene therapy DNA vector VTvaf17-IGF1 is extracted, multi-stage filtered, and purified by chromatographic methods. It is known to the experts in this field that culture conditions of strains, composition of nutrient media (except for antibiotic-free), equipment used, and DNA purification methods may vary within the framework of standard operating procedures depending on the particular production line, but known approaches to scaling, industrial production, and purification of DNA vectors using Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 fall within the scope of this invention.

The claimed disclosure of the invention is illustrated by examples of the embodiment of this invention.

The essence of the invention is explained in the following examples.

### Example 1

Production of gene therapy DNA vector VTvaf17-BDNF carrying the therapeutic gene, namely the BDNF gene.

Gene therapy DNA vector VTvaf17-BDNF was constructed by cloning the coding region of BDNF gene (750 bp) to a 3165 bp DNA vector VTvaf17 by BamHI and EcoRI restriction sites. The coding region of BDNF gene (750 bp) was obtained by isolating total RNA from the biological human tissue sample followed by reverse transcription reaction using commercial kit Mint-2 (Evrogen, Russia) and PCR amplification using the following oligonucleotides:

BDNF_F
GGATCCGCCACCATGACCATCCTTTTCCTTACTATG,
BDNF_R
AGGGAATTCCTATCTTCCCCTTTTAATGGTC

and commercially available kit Phusion® High-Fidelity DNA Polymerase (New England Biolabs, USA).

Gene therapy DNA vector VTvaf17 was constructed by consolidating six fragments of DNA derived from different sources:
(a) the origin of replication was produced by PCR amplification of a region of commercially available plasmid pBR322 with a point mutation,
(b) EF1a promoter region was produced by PCR amplification of a site of human genomic DNA,
(c) hGH-TA transcription terminator was produced by PCR amplification of a site of human genomic DNA,
(d) the RNA-OUT regulatory site of transposon Tn10 was synthesised from oligonucleotides,
(e) kanamycin resistance gene was produced by PCR amplification of a site of commercially available human plasmid pET-28,
(f) the polylinker was produced by annealing two synthetic oligonucleotides.

PCR amplification was performed using the commercially available kit Phusion® High-Fidelity DNA Polymerase (New England Biolabs, USA) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

The amplification product of the coding region of BDNF gene and DNA vector VTvaf17 was cleaved by BamHI and EcoRI restriction endonucleases (New England Biolabs, USA).

This resulted in a 3891 bp DNA vector VTvaf17-BDNF with the nucleotide sequence SEQ ID No. 1 and general structure shown in FIG. 1A.

### Example 2

Production of gene therapy DNA vector VTvaf17-VEGFA carrying the therapeutic gene, namely the VEGFA gene.

Gene therapy DNA vector VTvaf17-VEGFA was constructed by cloning the coding region of VEGFA gene (1242 bp) to a 3165 bp DNA vector VTvaf17 by BamHI and HindIII restriction sites. The coding region of VEGFA gene (1242 bp) was obtained by isolating total RNA from the biological human tissue sample followed by reverse transcription reaction using commercial kit Mint-2 (Evrogen, Russia) and PCR amplification using the following oligonucleotides:

VEGFA_F
GGGGGATCCACCATGACGGACAGACAGACAGACACCGC,
VEGFA_R
TTTGGATCCACCATGAACTTTCTGCTGTCTTGGGTGC

and commercially available kit Phusion® High-Fidelity DNA Polymerase (New England Biolabs, USA); amplification product and DNA vector VTvaf17 were cleaved by restriction endonucleases BamHI and HindIII (New England Biolabs, USA).

This resulted in a 4395 bp DNA vector VTvaf17-VEGFA with the nucleotide sequence SEQ ID No. 2 and general structure shown in FIG. 1B.

Gene therapy DNA vector VTvaf17 was constructed as described in Example 1.

### Example 3

Production of gene therapy DNA vector VTvaf17-BFGF carrying the therapeutic gene, namely the human BFGF gene.

Gene therapy DNA vector VTvaf17-BFGF was constructed by cloning the coding region of BFGF gene (872 bp) to a 3165 bp DNA vector VTvaf17 by HindIII, EcoRI restriction sites. The coding region of BFGF gene (872 bp) was obtained by isolating total RNA from the biological human tissue sample followed by reverse transcription reaction using commercial kit Mint-2 (Evrogen, Russia) and PCR amplification using the following oligonucleotides:

BFGF_F
GAGGAAGCTTCCACCATGGTGGGTGTGGGGGGTGGAGATG,
BFGF_R
GAGGGAATTCTCAGCTCTTAGCAGACATTGGAAGA

and commercially available kit Phusion® High-Fidelity DNA Polymerase (New England Biolabs, USA); amplification product and DNA vector VTvaf17 were cleaved by restriction endonucleases HindIII, EcoRI (New England Biolabs, USA).

This resulted in a 4031 bp DNA vector VTvaf17-BFGF with the nucleotide sequence SEQ ID No. 3 and general structure shown in FIG. 1C.

Gene therapy DNA vector VTvaf17 was constructed as described in Example 1.

### Example 4

Production of gene therapy DNA vector VTvaf17-NGF carrying the therapeutic gene, namely the NGF gene.

Gene therapy DNA vector VTvaf17-NGF was constructed by cloning the coding region of NGF gene (726 bp) to a 3165 bp DNA vector VTvaf17 by SaII and KpnI restriction sites. The coding region of NGF gene (726 bp) was obtained by isolating total RNA from the biological human tissue sample followed by reverse transcription reaction using commercial kit Mint-2 (Evrogen) and PCR amplification using the following oligonucleotides:

NGF_F
TTTGTCGACCACCATGTCCATGTTGTTCTACACTCTGATC,
NGF_R
AATGGTACCTCAGGCTCTTCTCACAGCCTTCC

and commercially available kit Phusion® High-Fidelity DNA Polymerase (New England Biolabs, USA); amplification product and DNA vector VTvaf17 were cleaved by restriction endonucleases SaII and KpnI (New England Biolabs, USA).

This resulted in a 3889 bp DNA vector VTvaf17-NGF with the nucleotide sequence SEQ ID No. 4 and general structure shown in FIG. 1D.

Gene therapy DNA vector VTvaf17 was constructed as described in Example 1.

### Example 5

Production of gene therapy DNA vector VTvaf17-GDNF carrying the therapeutic gene, namely the GDNF gene.

Gene therapy DNA vector VTvaf17-GDNF was constructed by cloning the coding region of GDNF gene (693 bp) to a 3165 bp DNA vector VTvaf17 by BamHI and HindIII restriction sites. The coding region of GDNF gene (693 bp) was obtained by isolating total RNA from the biological human tissue sample followed by reverse transcription reaction using commercial kit Mint-2 (Evrogen, Russia) and PCR amplification using the following oligonucleotides:

GDNF_F
GGGGGATCCACCATGCAGTCTTTGCCTAACAGCAATGG,
GDNF_R
TTTAAGCTTTCAGATACATCCACACCTTTTAGCG

and commercially available kit Phusion® High-Fidelity DNA Polymerase (New England Biolabs, USA); amplification product and DNA vector VTvaf17 were cleaved by restriction endonucleases BamHI and HindIII (New England Biolabs, USA).

This resulted in a 3846 bp DNA vector VTvaf17-GDNF with the nucleotide sequence SEQ ID No. 5 and general structure shown in FIG. 1E.

Gene therapy DNA vector VTvaf17 was constructed as described in Example 1.

### Example 6

Production of gene therapy DNA vector VTvaf17-NT3 carrying the therapeutic gene, namely the human NT3 gene.

Gene therapy DNA vector VTvaf17-NT3 was constructed by cloning the coding region of NT3 gene (816 bp) to a 3165 bp DNA vector VTvaf17 by BamHI and HindIII restriction sites. The coding region of NT3 gene (816 bp) was obtained by isolating total RNA from the biological human tissue sample followed by reverse transcription reaction using commercial kit Mint-2 (Evrogen, Russia) and PCR amplification using the following oligonucleotides:

NT3_F
AGGATCCACCATGGTTACTTTTGCCACGATC,
NT3_R
TATAAGCTTTCATGTTCTTCCGATTTTTCTC

and commercially available kit Phusion® High-Fidelity DNA Polymerase (New England Biolabs, USA); amplification product and DNA vector VTvaf17 were cleaved by restriction endonucleases BamHI and HindIII (New England Biolabs, USA).

This resulted in a 3969 bp DNA vector VTvaf17-NT3 with the nucleotide sequence SEQ ID No. 6 and general structure shown in FIG. 1F.

Gene therapy DNA vector VTvaf17 was constructed as described in Example 1.

### Example 7

Production of gene therapy DNA vector VTvaf17-CNTF carrying the therapeutic gene, namely the CNTF gene.

Gene therapy DNA vector VTvaf17-CNTF was constructed by cloning the coding region of CNTF gene (607 bp) to a 3165 bp DNA vector VTvaf17 by BamHII and HindIII restriction sites. The coding region of CNTF gene (607 bp) was obtained by isolating total RNA from the biological human tissue sample followed by reverse transcription reaction using commercial kit Mint-2 (Evrogen) and PCR amplification using the following oligonucleotides:

CNTF_F
TTTGTCGACCACCATGGCTTTCACAGAGCATTCACC,
CNTF_R
AATGGTACCTACATTTTCTTGTTGTTAGCAATATAATGG

and commercially available kit Phusion® High-Fidelity DNA Polymerase (New England Biolabs, USA); amplification product and DNA vector VTvaf17 were cleaved by restriction endonucleases BamHII and HindIII (New England Biolabs, USA).

This resulted in a 3765 bp DNA vector VTvaf17-CNTF with the nucleotide sequence SEQ ID No. 7 and general structure shown in FIG. 1G.

Gene therapy DNA vector VTvaf17 was constructed as described in Example 1.

### Example 8

Production of gene therapy DNA vector VTvaf17-IGF1 carrying the therapeutic gene, namely the IGF1 gene.

Gene therapy DNA vector VTvaf17-IGF1 was constructed by cloning the coding region of IGF1 gene (481 bp) to a 3165 bp DNA vector VTvaf17 by SaII and KpnI restriction sites. The coding region of IGF1 gene (481 bp) was obtained by isolating total RNA from the biological human tissue sample followed by reverse transcription reaction using commercial kit Mint-2 (Evrogen) and PCR amplification using the following oligonucleotides:

IGF1_F
TTTGTCGACCACCATGGGAAAAATCAGCAGTCTTCC,
IGF1_R
AATGGTACCTACTTGCGTTCTTCAAATGTACTTCC

and commercially available kit Phusion® High-Fidelity DNA Polymerase (New England Biolabs, USA); amplification product and DNA vector VTvaf17 were cleaved by restriction endonucleases SalI and KpnI (New England Biolabs, USA).

This resulted in a 3639 bp DNA vector VTvaf17-IGF1 with the nucleotide sequence SEQ ID No. 8 and general structure shown in FIG. 1H.

Gene therapy DNA vector VTvaf17 was constructed as described in Example 1.

### Example 9

Proof of the ability of gene therapy DNA vector VTvaf17-BDNF carrying the therapeutic gene, namely BDNF gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the BDNF therapeutic gene were assessed in HSkM human primary skeletal muscle myoblast cell culture (Gibco cat #A12555) 48 hours after its transfection with gene therapy DNA vector VTvaf17-BDNF carrying the human BDNF gene. The amount of mRNA was determined by the dynamics of accumulation of cDNA amplicons in the real-time PCR.

HSkM human primary skeletal muscle myoblast cell culture was used for the assessment of changes in the therapeutic BDNF mRNA accumulation. HSkM cell culture was grown under standard conditions (37° C., 5% CO2) using the DMEM growth medium. The growth medium was replaced every 48 hours during the cultivation process.

To achieve 90% confluence, 24 hours before the transfection procedure, the cells were seeded into a 24-well plate in the quantity of 5×104 cells per well. Transfection with gene therapy DNA vector VTvaf17-BDNF expressing the human BDNF gene was performed using Lipofectamine 3000 (ThermoFisher Scientific, USA) according to the manufacturer's recommendations. In test tube 1, 1 μl of DNA vector VTvaf17-BDNF solution (concentration 500 ng/μl) and 1 μl of reagent P3000 was added to 25 μl of medium Opti-MEM (Gibco, USA). The preparation was mixed by gentle shaking. In test tube 2, 1 μl of Lipofectamine 3000 solution was added to 25 μl of medium Opti-MEM (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

HSKM cells transfected with the gene therapy DNA vector VTvaf17 devoid of the inserted therapeutic gene (cDNA of BDNF gene before and after transfection with gene therapy DNA vector VTvaf17 devoid of the inserted therapeutic gene is not shown in the figures) were used as a reference. Reference vector VTvaf17 for transfection was prepared as described above.

Total RNA from HSKM cells was extracted using Trizol Reagent (Invitrogen, USA) according to the manufacturer's recommendations. 1 ml of Trizol Reagent was added to the well with cells, homogenised and heated for 5 minutes at 65° C. The sample was centrifuged at 14,000 g for 10 minutes and heated again for 10 minutes at 65° C. Then, 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried and dissolved in 10 μl of RNase-free water. The level of BDNF mRNA expression after transfection was determined by assessing the dynamics of the accumulation of cDNA amplicons by real-time PCR. For the production and amplification of cDNA specific for the human BDNF gene, the following BDNF_SF and BDNF SR oligonucleotides were used:

BDNF_SF
TTTGGTTGCATGAAGGCTGC,
BDNF_SR
GCCGAACTTTCTGGTCCTCA

The length of amplification product is 199 bp.

Reverse transcription reaction and PCR amplification was performed using SYBR GreenQuantitect RT-PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 30 s. B2M (beta-2-microglobuline) gene listed in the GenBank database under number NM 004048.2 was used as a reference gene. Positive control included amplicons from PCR on matrices represented by plasmids in known concentrations containing cDNA sequences of BDNF and B2M genes. Negative control included deionised water. Real-time quantification of the dynamics of accumulation of cDNA amplicons of BDNF and B2M genes was conducted using the Bio-Rad CFX Manager 2.1 software (Bio-Rad, USA). Diagrams resulting from the assay are shown in FIG. 2.

FIG. 2 shows that the level of specific mRNA of human BDNF gene has grown massively as a result of transfection of HSkM human skeletal myoblast cell culture with gene therapy DNA vector VTvaf17-BDNF, which confirms the ability of the vector to penetrate eukaryotic cells and express the BDNF gene at the mRNA level. The presented results also confirm the practicability of use of gene therapy DNA vector VTvaf17-BDNF in order to increase the expression level of BDNF gene in eukaryotic cells.

### Example 10

Proof of the ability of gene therapy DNA vector VTvaf17-VEGFA carrying the therapeutic gene, namely VEGFA gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the VEGFA therapeutic gene were assessed in HBdSMC primary human urinary bladder smooth muscle cells (ATCC PCS-420-012) 48 hours after their transfection with gene therapy DNA vector VTvaf17-VEGFA carrying the human VEGFA gene. The amount of mRNA was determined by the dynamics of accumulation of cDNA amplicons in the real-time PCR.

HBdSMC primary human urinary bladder smooth muscle cell culture was grown in the medium with growth additives prepared using the Vascular Smooth Muscle Cell GroCGRPh Kit (ATCC® PCS-100-042™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-VEGFA expressing the human VEGFA gene was performed according to the procedure described in Example 9. B2M (beta-2-microglobuline) gene listed in the GenBank database under number NM 004048.2 was used as a reference gene. HBdSMC cell culture transfected with the gene therapy DNA vector VTvaf17 devoid of the therapeutic gene (cDNA of VEGFA gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human VEGFA gene, the following VEGFA_SF and VEGFA_SR oligonucleotides were used:

VEGFA_SF
TCTGCTGTCTTGGGTGCATT,
VEGFA_SR
CCAGGGTCTCGATTGGATGG

The length of amplification product is 167 bp.

Positive control included amplicons from PCR on matrices represented by plasmids in known concentrations containing cDNA sequences of VEGFA and B2M genes. Negative control included deionised water. Real-time quantification of the PCR products, i.e. VEGFA and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that the level of specific mRNA of human VEGFA gene has grown massively as a result of transfection of HBdSMC primary human urinary bladder smooth muscle cell culture with gene therapy DNA vector VTvaf17-VEGFA, which confirms the ability of the vector to penetrate eukaryotic cells and express the VEGFA gene at the mRNA level. The presented results also confirm the practicability of use of gene therapy DNA vector VTvaf17-VEGFA in order to increase the expression level of VEGFA gene in eukaryotic cells.

### Example 11

Proof of the ability of gene therapy DNA vector VTvaf17-BFGF carrying the therapeutic gene, namely BFGF gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the BFGF therapeutic gene were assessed in T/G HA-VSMC primary human aortic smooth muscle cell culture (ATCC CRL-1999™) 48 hours after its transfection with gene therapy DNA vector VTvaf17-BFGF carrying the human BFGF gene. The amount of mRNA was determined by the dynamics of accumulation of cDNA amplicons in the real-time PCR.

T/G HA-VSMC primary human aortic smooth muscle cell culture was grown in F-12K Medium (ATCC) with the addition of 0.05 mg/ml ascorbic acid, 0.01 mg/ml insulin, 0.01 mg/ml transferrin, 10 ng/ml sodium selenite, 0.03 mg/ml Endothelial Cell Growth Supplement (ECGS), 10% fetal bovine serum under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-BFGF expressing the human BFGF gene was performed according to the procedure described in Example 9. B2M (beta-2-microglobuline) gene listed in the GenBank database under number NM 004048.2 was used as a reference gene. T/G HA-VSMC cell line transfected with the gene therapy DNA vector VTvaf17 devoid of the therapeutic gene (cDNA of BFGF gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human BFGF gene, the following BFGF_SF and BFGF_SR oligonucleotides were used:

BFGF_SF
TGTGCTAACCGTTACCTGGC,
BFGF_SR
ACTGCCCAGTTCGTTTCAGT

The length of amplification product is 166 bp.

Positive control included amplicons from PCR on matrices represented by plasmids in known concentrations containing cDNA sequences of BFGF and B2M genes. Negative control included deionised water. Real-time quantification of the PCR products, i.e. BFGF and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that the level of specific mRNA of human BFGF gene has grown massively as a result of transfection of T/G HA-VSMC primary human aortic smooth muscle cell culture with gene therapy DNA vector VTvaf17-BFGF, which confirms the ability of the vector to penetrate eukaryotic cells and express the BFGF gene at the mRNA level. The presented results also confirm the practicability of use of gene therapy DNA vector VTvaf17-BFGF in order to increase the expression level of BFGF gene in eukaryotic cells.

### Example 12

Proof of the ability of gene therapy DNA vector VTvaf17-NGF carrying the therapeutic gene, namely NGF gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the NGF therapeutic gene were assessed in HUVEC human umbilical vein endothelial cells (ATCC® PCS-100-013™) 48 hours after their transfection with gene therapy DNA vector VTvaf17-NGF carrying the human NGF gene. The amount of mRNA was determined by the dynamics of accumulation of cDNA amplicons in the real-time PCR.

HUVEC human umbilical vein endothelial cell culture was grown in Endothelial Cell Growth Kit-BBE medium (ATCC® PCS-100-040) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-NGF expressing the human NGF gene was performed according to the procedure described in Example 9. HUVEC cell culture transfected with the gene therapy DNA vector VTvaf17 devoid of the therapeutic gene (cDNA of NGF gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human NGF gene, the following NGF_SF and NGF_SR oligonucleotides were used:

NGF_SF
TGAAGCTGCAGACACTCAGG,
NGF_SR
CTCCCAACACCATCACCTCC

The length of amplification product is 200 bp.

Positive control included amplicons from PCR on matrices represented by plasmids in known concentrations containing cDNA sequences of NGF and B2M genes. B2M (beta-2-microglobuline) gene listed in the GenBank database under number NM 004048.2 was used as a reference gene. Negative control included deionised water. Real-time quantification of the PCR products, i.e. NGF and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that the level of specific mRNA of human NGF gene has grown massively as a result of transfection of HUVEC human umbilical vein endothelial cells with gene therapy DNA vector VTvaf17-NGF, which confirms the ability of the vector to penetrate eukaryotic cells and express the NGF gene at the mRNA level. The presented results also confirm the practicability of use of gene therapy DNA vector VTvaf17-NGF in order to increase the expression level of NGF gene in eukaryotic cells.

### Example 13

Proof of the ability of gene therapy DNA vector VTvaf17-GDNF carrying the therapeutic gene, namely GDNF gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the therapeutic GDNF gene were assessed in HMEC-1 human dermal microvascular endothelial cell line (ATCC CRL-3243) 48 hours after its transfection with gene therapy DNA vector VTvaf17-GDNF carrying the human GDNF gene. The amount of mRNA was determined by the dynamics of accumulation of cDNA amplicons in the real-time PCR.

HMEC-1 human dermal microvascular endothelial cell culture was grown in MCDB131 medium (Gibco™, Cat. 10372019) without glutamine and with the addition of 10 ng/ml of recombinant EGF (Sigma, E9644, USA), 10 mM glutamine (Paneco, Russia), 1 μg/ml hydrocortisone (Sigma H0888, USA), 10% HyClone™ Fetal Bovine Serum (Hyclone Laboratories Inc SH30068.03HI, USA) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-GDNF expressing the human GDNF gene was performed according to the procedure described in Example 9. B2M (beta-2-microglobuline) gene listed in the GenBank database under number NM 004048.2 was used as a reference gene. HMEC-1 cell culture transfected with the gene therapy DNA vector VTvaf17 devoid of the therapeutic gene (cDNA of GDNF gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human GDNF gene, the following GDNF_SF and GDNF SR oligonucleotides were used:

GDNF_SF
GTCACTGACTTGGGTCTGGG,
GDNF_SR
GCCTGCCCTACTTTGTCACT

The length of amplification product is 152 bp.

Positive control included amplicons from PCR on matrices represented by plasmids in known concentrations containing cDNA sequences of GDNF and B2M genes. Negative control included deionised water. Real-time quantification of the PCR products, i.e. GDNF and B2M gene cDNAs obtained by amplification, was conducted using the Bio-Rad CFX Manager 2.1 software (Bio-Rad, USA). Diagrams resulting from the assay are shown in FIG. 6.

FIG. 6 shows that the level of specific mRNA of human GDNF gene has grown massively as a result of transfection of HMEC-1 human dermal microvascular endothelial cell culture with gene therapy DNA vector VTvaf17-GDNF, which confirms the ability of the vector to penetrate eukaryotic cells and express the GDNF gene at the mRNA level. The presented results also confirm the practicability of use of gene therapy DNA vector VTvaf17-GDNF in order to increase the expression level of GDNF gene in eukaryotic cells.

### Example 14

Proof of the ability of gene therapy DNA vector VTvaf17-NT3 carrying the therapeutic gene, namely NT3 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the NT3 therapeutic gene were assessed in SH-SY5Y human neuroblastoma cells (ATCC® CRL-2266™) 48 hours after their transfection with gene therapy DNA vector VTvaf17-NT3 carrying the human NT3 gene. The amount of mRNA was determined by the dynamics of accumulation of cDNA amplicons in the real-time PCR.

SH-SY5Y human neuroblastoma cell culture was grown in medium using a mixture of the following growth media (1:1): ATCC-formulated Eagle's Minimum Essential Medium (ATCC, 30-2003) and F12 Medium (ATCC® 30-2006™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-NT3 expressing the human NT3 gene was performed according to the procedure described in Example 9. B2M (beta-2-microglobuline) gene listed in the GenBank database under number NM 004048.2 was used as a reference gene. SH-SY5Y cell culture transfected with the gene therapy DNA vector VTvaf17 devoid of the therapeutic gene (cDNA of NT3 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human NT3 gene, the following NT3_SF and NT3_SR oligonucleotides were used:

NT3_SF
AACTGCTGCGACAACAGAGA,
NT3_SR
GTACTCCCCTCGGTGACTCT

The length of amplification product is 176 bp.

Positive control included amplicons from PCR on matrices represented by plasmids in known concentrations containing cDNA sequences of NT3 and B2M genes. Negative control included deionised water. Real-time quantification of the PCR products, i.e. NT3 and B2M gene cDNAs obtained by amplification, was conducted using the Bio-Rad CFX Manager 2.1 software (Bio-Rad, USA). Diagrams resulting from the assay are shown in FIG. 7.

FIG. 7 shows that the level of specific mRNA of human NT3 gene has grown massively as a result of transfection of SH-SY5Y human neuroblastoma cell culture with gene therapy DNA vector VTvaf17-NT3, which confirms the ability of the vector to penetrate eukaryotic cells and express the NT3 gene at the mRNA level. The presented results also confirm the practicability of use of gene therapy DNA vector VTvaf17-NT3 in order to increase the expression level of NT3 gene in eukaryotic cells.

### Example 15

Proof of the ability of gene therapy DNA vector VTvaf17-CNTF carrying the therapeutic gene, namely CNTF gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the CNTF therapeutic gene were assessed in primary human corneal epithelial cell culture (ATCC® PCS-700-010™) 48 hours after its transfection with gene therapy DNA vector VTvaf17-CNTF carrying the human CNTF gene. The amount of mRNA was determined by the dynamics of accumulation of cDNA amplicons in the real-time PCR.

Primary human corneal epithelial cell culture was grown in Corneal Epithelial Cell Basal Medium (ATCC® PCS-700-030™) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-CNTF expressing the human CNTF gene was performed according to the procedure described in Example 9. Primary human corneal epithelial cell culture transfected with the gene therapy DNA vector VTvaf17 not carrying the therapeutic gene (cDNA of CNTF gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human CNTF gene, the following CNTF_SF and CNTF_SR oligonucleotides were used:

CNTF_SF
ACATCAACCTGGACTCTGCG,
CNTF_SR
TGGAAGTCACCTTCGGTTGG

The length of amplification product is 178 bp.

Positive control included amplicons from PCR on matrices represented by plasmids in known concentrations containing cDNA sequences of CNTF and B2M genes. B2M (beta-2-microglobuline) gene listed in the GenBank database under number NM 004048.2 was used as a reference gene. Negative control included deionised water. Real-time quantification of the PCR products, i.e. CNTF and B2M gene cDNAs obtained by amplification, was conducted using the Bio-Rad CFX Manager 2.1 software (Bio-Rad, USA). Diagrams resulting from the assay are shown in FIG. 8.

FIG. 8 shows that the level of specific mRNA of human CNTF gene has grown massively as a result of transfection of primary human corneal epithelial cell culture with gene therapy DNA vector VTvaf17-CNTF, which confirms the ability of the vector to penetrate eukaryotic cells and express the CNTF gene at the mRNA level. The presented results also confirm the practicability of use of gene therapy DNA vector VTvaf17-CNTF in order to increase the expression level of CNTF gene in eukaryotic cells.

### Example 16

Proof of the ability of gene therapy DNA vector VTvaf17-IGF1 carrying the therapeutic gene, namely IGF1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the IGF1 therapeutic gene were assessed in HMEC human mammary epithelial cell culture (ATCC® PCS-600-010™) 48 hours after its transfection with gene therapy DNA vector VTvaf17-IGF1 carrying the human IGF1 gene. The amount of mRNA was determined by the dynamics of accumulation of cDNA amplicons in the real-time PCR.

HMEC human mammary epithelial cell culture was grown in Mammary Epithelial Cell Basal Medium (PCS-600-030) with the addition of Mammary Epithelial Cell Growth Kit (PCS-600-040) under standard conditions (37° C., 5% CO2). To achieve 90% confluence, 24 hours before the transfection procedure, the cells were seeded into a 24-well plate in the quantity of 5×104 cells per well. Lipofectamine 3000 (ThermoFisher Scientific, USA) was used as a transfection reagent. The transfection with gene therapy DNA vector VTvaf17-IGF1 expressing the human IGF1 gene was performed according to the procedure described in Example 9. HMEC cell culture transfected with the gene therapy DNA vector VTvaf17 devoid of the therapeutic gene (cDNA of IGF1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human IGF1 gene, the following IGF1_SF and IGF1_SR oligonucleotides were used:

IGF1_SF
CCATGTCCTCCTCGCATCTC,
IGF1_SR
ACCCTGTGGGCTTGTTGAAA.

The length of amplification product is 159 bp.

Positive control included amplicons from PCR on matrices represented by plasmids in known concentrations containing cDNA sequences of IGF1 and B2M genes. B2M (beta-2-microglobuline) gene listed in the GenBank database under number NM 004048.2 was used as a reference gene. Negative control included deionised water. Real-time quantification of the PCR products, i.e. IGF1 and B2M gene cDNAs obtained by amplification, was conducted using the Bio-Rad CFX Manager 2.1 software (Bio-Rad, USA). Diagrams resulting from the assay are shown in FIG. 9.

FIG. 9 shows that the level of specific mRNA of human IGF1 gene has grown massively as a result of transfection of HMEC human mammary epithelial cell culture with gene therapy DNA vector VTvaf17-IGF1, which confirms the ability of the vector to penetrate eukaryotic cells and express the IGF1 gene at the mRNA level. The presented results also confirm the practicability of use of gene therapy DNA vector VTvaf17-IGF1 in order to increase the expression level of IGF1 gene in eukaryotic cells.

### Example 17

Proof of the efficiency and practicability of use of gene therapy DNA vector VTvaf17-BDNF carrying the BDNF gene in order to increase the expression of BDNF protein in mammalian cells.

The change in the BDNF protein concentration in the lysate of HSKM human primary skeletal muscle myoblast cells (Gibco cat #A12555) after transfection of these cells with DNA vector VTvaf17-BDNF carrying the human BDNF gene was assessed.

HSkM human primary skeletal muscle myoblast cell culture was grown as described in Example 9.

To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector VTvaf17 devoid of cDNA of BDNF gene (B) were used as a reference, and DNA vector VTvaf17-BDNF carrying the human BDNF gene (C) was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, the culture medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The BDNF protein was assayed by enzyme-linked immunosorbent assay (ELISA) using the Human BDNF ELISA Kit (Sandwich ELISA) (LifeSpan BioSciences LS-F35-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of BDNF protein was used. The sensitivity was at least 80 pg/ml, measurement range—from 66 pg/ml to 16000 pg/ml. R-3.0.2 was used for the statistical treatment of the results and data visualization (https://www.r-project.org/). Drawings resulting from the assay are shown in FIG. 10.

FIG. 10 shows that the transfection of HSkM primary human skeletal myoblast cell culture with gene therapy DNA vector VTvaf17-BDNF results in increased BDNF protein concentration compared to reference samples, which confirms the ability of the vector to penetrate eukaryotic cells and express the BDNF gene at the protein level. The presented results also confirm the practicability of use of gene therapy DNA vector VTvaf17-BDNF in order to increase the expression level of BDNF gene in eukaryotic cells.

### Example 18

Proof of the efficiency and practicability of use of gene therapy DNA vector VTvaf17-VEGFA carrying the VEGFA gene in order to increase the expression of VEGFA protein in mammalian cells.

The change in the VEGFA protein concentration in the cell lysate of HBdSMC primary human urinary bladder smooth muscle cell culture (ATCC PCS-420-012) was assessed after transfection of these cells with the DNA vector VTvaf17-VEGFA carrying the human VEGFA gene. Cells were grown as described in Example 10.

The 6th generation SuperFect Transfection Reagent (Qiagen, Germany) was used for transfection. The aqueous dendrimer solution without DNA vector (A) and DNA vector VTvaf17 devoid of cDNA of VEGFA gene (B) were used as a reference, and DNA vector VTvaf17-VEGFA carrying the human VEGFA gene (C) was used as the transfected agent. Preparation of the DNA dendrimer complex and transfection of HBdSMC cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The VEGFA protein was assayed by enzyme-linked immunosorbent assay (ELISA) using the Human VEGFA ELISA Kit (Sandwich ELISA) (LifeSpan BioSciences LS-F968-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of VEGFA protein was used. The sensitivity was at least 16 pg/ml, measurement range—from 16 pg/ml to 1000 pg/ml. R-3.0.2 was used for the statistical treatment of the results and data visualization (https://www.r-project.org/). Drawings resulting from the assay are shown in FIG. 11.

FIG. 11 shows that the transfection of HBdSMC human urinary bladder smooth muscle cell culture with gene therapy DNA vector VTvaf17-VEGFA results in increased VEGFA protein concentration compared to reference samples, which confirms the ability of the vector to penetrate eukaryotic cells and express the VEGFA gene at the protein level. The presented results also confirm the practicability of use of gene therapy DNA vector VTvaf17-VEGFA in order to increase the expression level of VEGFA gene in eukaryotic cells.

### Example 19

Proof of the efficiency and practicability of use of gene therapy DNA vector VTvaf17-BFGF carrying the BFGF gene in order to increase the expression of BFGF protein in mammalian cells.

The change in the BFGF protein concentration in the cell lysate of T/GHA-VSMC primary aortic smooth muscle cell culture (ATCC CRL-1999™) was assessed after transfection of these cells with the DNA vector VTvaf17-BFGF carrying the human BFGF gene. Cells were cultured as described in Example 11.

The 6th generation SuperFect Transfection Reagent (Qiagen, Germany) was used for transfection. The aqueous dendrimer solution without DNA vector (A) and DNA vector VTvaf17 devoid of cDNA of BFGF gene (B) were used as a reference, and DNA vector VTvaf17-BFGF carrying the human BFGF gene (C) was used as the transfected agent. Preparation of the DNA dendrimer complex and transfection of T/G HA-VSMC cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The BFGF protein was assayed by enzyme-linked immunosorbent assay (ELISA) using the Human FGF2/Basic FGF ELISA Kit (Sandwich ELISA) (LifeSpan BioSciences LS-F955) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of BFGF protein was used. The sensitivity was at least 63 pg/ml, measurement range—from 63 pg/ml to 400 pg/ml. R-3.0.2 was used for the statistical treatment of the results and data visualization (https://www.r-project.org/). Drawings resulting from the assay are shown in FIG. 12.

FIG. 12 shows that the transfection of T/GHA-VSMC primary aortic smooth muscle cells with gene therapy DNA vector VTvaf17-BFGF results in increased BFGF protein concentration compared to reference samples, which confirms the ability of the vector to penetrate eukaryotic cells and express the BFGF gene at the protein level. The presented results also confirm the practicability of use of gene therapy DNA vector VTvaf17-BFGF in order to increase the expression level of BFGF gene in eukaryotic cells.

### Example 20

Proof of the efficiency and practicability of use of gene therapy DNA vector VTvaf17-NGF carrying the NGF gene in order to increase the expression of NGF protein in mammalian cells.

Changes in the NGF protein concentration in the lysate of HUVEC human umbilical vein endothelial cells (ATCC® PCS-100-013™) were assessed after transfection of these cells with gene therapy DNA vector VTvaf17-NGF carrying the human NGF gene. Cells were cultured as described in Example 12.

The 6th generation SuperFect Transfection Reagent (Qiagen, Germany) was used for transfection. The aqueous dendrimer solution without DNA vector (A) and DNA vector VTvaf17 devoid of cDNA of NGF gene (B) were used as a reference, and DNA vector VTvaf17-NGF carrying the human NGF gene (C) was used as the transfected agent. Preparation of the DNA dendrimer complex and transfection of HUVEC cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The NGF protein was assayed by enzyme-linked immunosorbent assay (ELISA) using the Human NGF ELISA Kit (Sandwich ELISA) (LifeSpan BioSciences LS-F9557-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NGF protein was used. The sensitivity was at least 3.12 pg/ml, measurement range—from 3.12 pg/ml to 200 pg/ml. R-3.0.2 was used for the statistical treatment of the results and data visualization (https://www.r-project.org/). Drawings resulting from the assay are shown in FIG. 13.

FIG. 13 shows that the transfection of HUVEC human umbilical vein endothelial cell culture with gene therapy DNA vector VTvaf17-NGF results in increased NGF protein concentration compared to reference samples, which confirms the ability of the vector to penetrate eukaryotic cells and express the NGF gene at the protein level. The presented results also confirm the practicability of use of gene therapy DNA vector VTvaf17-NGF in order to increase the expression level of NGF gene in eukaryotic cells.

### Example 21

Proof of the efficiency and practicability of use of gene therapy DNA vector VTvaf17-GDNF carrying the GDNF gene in order to increase the expression of GDNF protein in mammalian cells.

Changes in the GDNF protein concentration in conditioned medium lysate of HMEC-1 human dermal microvascular endothelial cell line (ATCC CRL-3243) were assessed after transfection of these cells with gene therapy DNA vector VTvaf17-GDNF carrying the human GDNF gene. Cells were grown as described in Example 13.

The 6th generation SuperFect Transfection Reagent (Qiagen, Germany) was used for transfection. The aqueous dendrimer solution without DNA vector (A) and DNA vector VTvaf17 devoid of cDNA of GDNF gene (B) were used as a reference, and DNA vector VTvaf17-GDNF carrying the human GDNF gene (C) was used as the transfected agent. Preparation of the DNA dendrimer complex and transfection of HMEC-1 cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The GDNF protein was assayed by enzyme-linked immunosorbent assay (ELISA) using the Human GDNF ELISA Kit (Sandwich ELISA) (LifeSpan BioSciences LS-F2435) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of GDNF protein was used. The sensitivity was at least 4 pg/ml, measurement range—from 31.2 pg/ml to 2000 pg/ml. R-3.0.2 was used for the statistical treatment of the results and data visualization (https://www.r-project.org/). Drawings resulting from the assay are shown in FIG. 14.

FIG. 14 shows that the transfection of HMEC-1 human dermal microvascular endothelial cell line with gene therapy DNA vector VTvaf17-GDNF results in increased GDNF protein concentration compared to reference samples, which confirms the ability of the vector to penetrate eukaryotic cells and express the GDNF gene at the protein level. The presented results also confirm the practicability of use of gene therapy DNA vector VTvaf17-GDNF in order to increase the expression level of GDNF gene in eukaryotic cells.

### Example 22

Proof of the efficiency and practicability of use of gene therapy DNA vector VTvaf17-NT3 carrying the NT3 gene in order to increase the expression of NT3 protein in mammalian cells.

Changes in the NT3 protein concentration in the lysate of SH-SY5Y human neuroblastoma cell culture (ATCC® CRL-2266™) were assessed after transfection of these cells with gene therapy DNA vector VTvaf17-NT3 carrying the human NT3 gene. Cells were cultured as described in Example 14.

The 6th generation SuperFect Transfection Reagent (Qiagen, Germany) was used for transfection. The aqueous dendrimer solution without DNA vector (A) and DNA vector VTvaf17 devoid of cDNA of NT3 gene (B) were used as a reference, and DNA vector VTvaf17-NT3 carrying the human NT3 gene (C) was used as the transfected agent. Preparation of the DNA dendrimer complex and transfection of SH-SY5Y cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The NT3 protein was assayed by enzyme-linked immunosorbent assay (ELISA) using the Human NT-3 ELISA (RayBiotech ELH-NT3-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of NT3 protein was used. The sensitivity was at least 4 pg/ml, measurement range—from 4 pg/ml to 3000 pg/ml. R-3.0.2 was used for the statistical treatment of the results and data visualization (https://www.r-project.org/). Drawings resulting from the assay are shown in FIG. 15.

FIG. 15 shows that the transfection of SH-SY5Y human neuroblastoma cell culture with gene therapy DNA vector VTvaf17-NT3 results in increased NT3 protein concentration compared to reference samples, which confirms the ability of the vector to penetrate eukaryotic cells and express the NT3 gene at the mRNA level. The presented results also confirm the practicability of use of gene therapy DNA vector VTvaf17-NT3 in order to increase the expression level of NT3 gene in eukaryotic cells.

### Example 23

Proof of the efficiency and practicability of use of gene therapy DNA vector VTvaf17-CNTF carrying the CNTF gene in order to increase the expression of CNTF protein in mammalian cells.

The change in the CNTF protein concentration in the lysate of primary corneal epithelial cell culture (ATCC® PCS-700-010™) after transfection of these cells with DNA vector VTvaf17-CNTF carrying the human CNTF gene was assessed. Cells were cultured as described in Example 15.

The 6th generation SuperFect Transfection Reagent (Qiagen, Germany) was used for transfection. The aqueous dendrimer solution without DNA vector (A) and DNA vector VTvaf17 devoid of cDNA of CNTF gene (B) were used as a reference, and DNA vector VTvaf17-CNTF carrying the human CNTF gene (C) was used as the transfected agent. Preparation of the DNA dendrimer complex and transfection of corneal epithelial cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The CNTF protein was assayed by enzyme-linked immunosorbent assay (ELISA) using the Human CNTF ELISA Kit (Sandwich ELISA) (LifeSpan BioSciences LS-F3977-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of CNTF protein was used. The sensitivity was at least 3.2 pg/ml, measurement range—from 7.81 pg/ml to 500 pg/ml. R-3.0.2 was used for the statistical treatment of the results and data visualization (https://www.r-project.org/). Diagrams resulting from the assay are shown in FIG. 16.

FIG. 16 shows that the transfection of primary corneal epithelial cell culture with gene therapy DNA vector VTvaf17-CNTF results in increased CNTF protein concentration compared to reference samples, which confirms the ability of the vector to penetrate eukaryotic cells and express the CNTF gene at the mRNA level. The presented results also confirm the practicability of use of gene therapy DNA vector VTvaf17-CNTF in order to increase the expression level of CNTF gene in eukaryotic cells.

### Example 24

Proof of the efficiency and practicability of use of gene therapy DNA vector VTvaf17-IGF1 carrying the IGF1 gene in order to increase the expression of IGF1 protein in mammalian cells.

The change in the IGF1 protein concentration in the lysate of HMEC human mammary epithelial cell culture (ATCC® PCS-600-010™) after transfection of these cells with DNA vector VTvaf17-IGF1 carrying the human IGF1 gene was assessed. Cells were cultured as described in Example 16.

The 6th generation SuperFect Transfection Reagent (Qiagen, Germany) was used for transfection. The aqueous dendrimer solution without DNA vector (A) and DNA vector VTvaf17 devoid of cDNA of IGF1 gene (B) were used as a reference, and DNA vector VTvaf17-IGF1 carrying the human IGF1 (C) gene was used as the transfected agent. Preparation of the DNA dendrimer complex and transfection of HMEC cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The IGF1 protein was assayed by enzyme-linked immunosorbent assay (ELISA) using the Human IGF1 ELISA Kit (Sandwich ELISA) (LifeSpan BioSciences LS-F11726) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of IGF1 protein was used. The sensitivity was at least 78 pg/ml, measurement range—from 78 pg/ml to 5000 pg/ml. R-3.0.2 was used for the statistical treatment of the results and data visualization (https://www.r-project.org/). Diagrams resulting from the assay are shown in FIG. 17.

FIG. 17 shows that the transfection of HMEC primary human mammary epithelial cell culture with gene therapy DNA vector VTvaf17-IGF1 results in increased IGF1 protein concentration compared to reference samples, which confirms the ability of the vector to penetrate eukaryotic cells and express the IGF1 gene at the protein level. The presented results also confirm the practicability of use of gene therapy DNA vector VTvaf17-IGF1 in order to increase the expression level of IGF1 gene in eukaryotic cells.

### Example 25

Proof of the efficiency and practicability of use of gene therapy DNA vector VTvaf17-GDNF carrying the GDNF gene in order to increase the expression of GDNF protein in human tissues.

To confirm the efficiency of gene therapy DNA vector VTvaf17-GDNF carrying the therapeutic gene, namely the GDNF gene, and practicability of its use, changes in GDNF protein concentration in human skin upon injection of gene therapy DNA vector VTvaf17-GDNF carrying the human GDNF gene were assessed.

To analyse changes in the GDNF protein concentration, gene therapy DNA vector VTvaf17-GDNF carrying the GDNF gene was injected into the forearm skin of three patients with concurrent injection of a placebo being gene therapy DNA vector VTvaf17 devoid of cDNA of GDNF gene.

Patient 1, woman, 43 y.o. (P1); Patient 2, woman, 62 y.o. (P2); Patient 3, man, 49 y.o. (P3). Polyethyleneimine Transfection reagent cGMP grade in-vivo-jetPEI (Polyplus Transfection, France) was used as a transport system. Gene therapy DNA vector VTvaf17-GDNF containing cDNA of GDNF gene and gene therapy DNA vector VTvaf17 used as a placebo not containing cDNA of GDNF gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-GDNF carrying the GDNF gene were injected in the quantity of 1 mg for each genetic construct using the tunnel method with a 30 G needle to the depth of 3 mm. The injectate volume of gene therapy DNA vector VTvaf17 (placebo) and gene therapy DNA vector VTvaf17-GDNF carrying the GDNF gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-GDNF carrying the GDNF gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein by enzyme-linked immunosorbent assay (ELISA) using the Human GDNF ELISA Kit (Sandwich ELISA) (LifeSpan BioSciences LS-F2435) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of GDNF protein was used. The sensitivity was at least 4 pg/ml, measurement range—from 31.2 pg/ml to 2000 pg/ml. R-3.0.2 was used for the statistical treatment of the results and data visualization (https://www.r-project.org/). Drawings resulting from the assay are shown in FIG. 18.

FIG. 18 shows the increased GDNF protein concentration in the skin of all three patients in the injection site of gene therapy DNA vector VTvaf17-GDNF carrying the human GDNF therapeutic gene compared to the GDNF protein concentration in the injection site of gene therapy DNA vector VTvaf17 (placebo) devoid of the human GDNF gene, which indicates the efficiency of gene therapy DNA vector VTvaf17-GDNF and confirms the practicability of its use, in particular upon intracutaneous injection of gene therapy DNA vector in human tissues.

### Example 26

Proof of the efficiency and practicability of use of gene therapy DNA vector VTvaf17-BDNF carrying the BDNF gene in order to increase the expression of BDNF protein in human tissues.

To confirm the efficiency of gene therapy DNA vector VTvaf17-BDNF carrying the BDNF therapeutic gene and practicability of its use, the change in the BDNF protein concentration in human muscle tissues upon injection of gene therapy DNA vector VTvaf17-BDNF carrying the therapeutic gene, namely the human BDNF gene, was assessed.

To analyse changes in the concentration of BDNF protein, gene therapy DNA vector VTvaf17-BDNF carrying the BDNF gene with transport molecule was injected into the gastrocnemius muscle of three patients with concurrent injection of a placebo being gene therapy DNA vector VTvaf17 devoid of cDNA of BDNF gene with transport molecule.

Patient 1, man, 60 y.o. (P1); Patient 2, woman, 52 y.o. (P2); Patient 3, man, 57 y.o. (P3). Polyethyleneimine Transfection reagent cGMP grade in-vivo-jetPEI (Polyplus Transfection, France) was used as a transport system; sample preparation was carried out in accordance with the manufacturer's recommendations.

Gene therapy DNA vector VTvaf17 (placebo) and gene therapy DNA vector VTvaf17-BDNF carrying the BDNF gene were injected in the quantity of 1 mg for each genetic construct using the tunnel method with a 30 G needle to the depth of around 10 mm. The injectate volume of gene therapy DNA vector VTvaf17 (placebo) and gene therapy DNA vector VTvaf17-BDNF carrying the BDNF gene was 0.3 ml for each genetic construct. The points of injection of each genetic construct were located medially at 8 to 10 cm intervals.

The biopsy samples were taken on the 2nd day after the injection of the genetic constructs of gene therapy DNA vectors. The biopsy samples were taken from the patients' muscle tissues in the site of injection of gene therapy DNA vector VTvaf17-BDNF carrying the BDNF gene (I), gene therapy DNA vector VTvaf17 (placebo) (II), and intact site of gastrocnemius muscle (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The BDNF protein was assayed by enzyme-linked immunosorbent assay (ELISA) using the Human BDNF ELISA Kit (Sandwich ELISA) (LifeSpan BioSciences LS-F35-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of BDNF protein was used. The sensitivity was at least 80 pg/ml, measurement range—from 66 pg/ml to 16000 pg/ml. R-3.0.2 was used for the statistical treatment of the results and data visualization (https://www.r-project.org/). Drawings resulting from the assay are shown in FIG. 19.

FIG. 19 shows the increased BDNF protein concentration in the gastrocnemius muscle of all three patients in the injection site of gene therapy DNA vector VTvaf17-BDNF carrying the therapeutic gene, namely BDNF gene, compared to the BDNF protein concentration in the injection site of gene therapy DNA vector VTvaf17 (placebo) devoid of the human BDNF gene, which indicates the efficiency of gene therapy DNA vector VTvaf17-BDNF and confirms the practicability of its use, in particular upon intramuscular injection of gene therapy DNA vector in human tissues.

### Example 27

Proof of the efficiency and practicability of combined use of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene for the increase of the expression level of GDNF, NT3, CNTF, and IGF1 proteins in human tissues.

To prove the efficiency of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene and practicability of its use, the change in the GDNF, NT3, CNTF, and IGF1 protein concentration in human skin with concurrent injection of a mixture of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene was assessed.

To analyse changes in the GDNF, NT3, CNTF, and IGF1 protein concentration, a mixture of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene was injected into the forearm skin of three patients with concurrent injection of a placebo being gene therapy DNA vector VTvaf17 devoid of cDNA of GDNF, NT3, CNTF, and IGF1 gene.

Patient 1, man, 38 y.o. (P1); Patient 2, woman, 43 y.o. (P2); Patient 3, man, 48 y.o. (P3). Polyethyleneimine Transfection reagent cGMP grade in-vivo-jetPEI (Polyplus Transfection, France) was used as a transport system. A mixture (in the ratio of 1:1:1:1) of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, gene therapy DNA vector VTvaf17-IGF1 carrying the IGF1 gene, and gene therapy DNA vector VTvaf17 used as a placebo that does not contain the cDNA of GDNF, NT3, CNTF, and IGF1 genes each dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and a mixture of gene therapy DNA vector VTvaf17-GDNF, gene therapy DNA vector VTvaf17-NT3, gene therapy DNA vector VTvaf17-CNTF, and gene therapy DNA vector VTvaf17-IGF1 were injected in the quantity of 4 mg for each genetic construct using the tunnel method with a 30 G needle to the depth of 3 mm. The injectate volume of gene therapy DNA vector VTvaf17 (placebo) and a mixture of gene therapy DNA vector VTvaf17-GDNF, gene therapy DNA vector VTvaf17-NT3, gene therapy DNA vector VTvaf17-CNTF, and gene therapy DNA vector VTvaf17-IGF1 was 1.2 ml for each genetic construct. The points of injection of each genetic construct were located at 8 to 10 cm intervals at the forearm skin site.

The biopsy samples were taken on the 2nd day after the injection of gene therapy DNA vectors. The biopsy samples were taken from the patients' skin in the site of injection of a mixture of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 21 (quantification of GDNF protein), Example 22 (quantification of NT3 protein), and Example 23 (quantification of CNTF protein), Example 24 (quantification of IGF1 protein).

To measure the numerical value of concentration, the calibration curve constructed using the reference samples from each kit with known concentrations of GDNF, NT3, CNTF, and IGF1 protein was used. R-3.0.2 was used for the statistical treatment of the results and data visualization (https://www.r-project.org/). Drawings resulting from the assay are shown in FIG. 20.

FIG. 20 shows an increase in the concentration of GDNF, NT3, CNTF, and IGF1 protein in the skin of all three patients in the injection site of a mixture of gene therapy DNA vector VTvaf17-GDNF carrying the GDNF gene, gene therapy DNA vector VTvaf17-NT3 carrying the NT3 gene, gene therapy DNA vector VTvaf17-CNTF carrying the CNTF gene, and gene therapy DNA vector VTvaf17-IGF1 carrying the IGF1 gene, compared to the GDNF, NT3, CNTF, and IGF1 protein concentration in the injection site of gene therapy DNA vector VTvaf17 (placebo) devoid of the human GDNF, NT3, CNTF, and IGF1 gene, which indicates the efficiency of gene therapy DNA vector VTvaf17-GDNF, gene therapy DNA vector VTvaf17-NT3, gene therapy DNA vector VTvaf17-CNTF, and gene therapy DNA vector VTvaf17-IGF1 and confirms the practicability of its use, in particular upon intracutaneous injection of gene therapy DNA vector in human tissues.

### Example 28

Proof of the efficiency of gene therapy DNA vector VTvaf17-VEGFA carrying the VEGFA gene and practicability of its use in order to increase the expression level of the VEGFA protein in human tissues by injecting autologous fibroblasts transfected with gene therapy DNA vector VTvaf17-VEGFA.

To confirm the efficiency of gene therapy DNA vector VTvaf17-VEGFA carrying the VEGFA gene and practicability of its use, changes in the VEGFA protein concentration in patient's skin upon injection of autologous fibroblast culture of the same patient transfected with gene therapy DNA vector VTvaf17-VEGFA were assessed.

The appropriate autologous fibroblast culture transfected with the gene therapy DNA vector VTvaf17-VEGFA carrying the VEGFA gene was injected into the patient's forearm skin with concurrent injection of a placebo in the form of autologous fibroblast culture transfected with gene therapy DNA vector VTvaf17 not carrying the VEGFA gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10 mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×10⁴ cells was taken from the cell culture. The patient's fibroblast culture was transfected with the gene therapy DNA vector VTvaf17-VEGFA carrying the VEGFA gene or placebo, i.e. VTvaf17 vector not carrying the VEGFA therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-VEGFA and autologous fibroblast culture of the patient transfected with gene therapy DNA vector VTvaf17 as a placebo was performed in the forearm using the tunnel method with a 13 mm long 30 G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after the injection of autologous fibroblast culture transfected with the gene therapy DNA vector VTvaf17-VEGFA carrying the therapeutic gene, namely VEGFA gene, and placebo. Biopsy was taken from the patient's skin in the site of injection of autologous fibroblast culture transfected with gene therapy DNA vector VTvaf17-VEGFA carrying the therapeutic gene, namely VEGFA gene (C), autologous fibroblast culture transfected with gene therapy DNA vector VTvaf17 not carrying the VEGFA therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the VEGFA therapeutic protein as described in Example 18.

Drawings resulting from the assay are shown in FIG. 21.

FIG. 21 shows the increased concentration of VEGFA protein in the area of the patient's skin in the injection site of autologous fibroblast culture transfected with the gene therapy DNA vector VTvaf17-VEGFA carrying the VEGFA gene compared to the VEGFA protein concentration in the injection site of autologous fibroblast culture transfected with the gene therapy DNA vector VTvaf17 that does not carry the VEGFA gene (placebo), which indicates the efficiency of gene therapy DNA vector VTvaf17-VEGFA and practicability of its use in order to increase the expression level of VEGFA in human tissues, in particular upon injection of autologous fibroblasts transfected with the gene therapy DNA vector VTvaf17-VEGFA into the skin.

### Example 29

Proof of the efficiency and practicability of combined use of gene therapy DNA vector VTvaf17-BDNF carrying the BDNF gene, gene therapy DNA vector VTvaf17-VEGFA carrying the VEGFA gene, gene therapy DNA vector VTvaf17-BFGF carrying the BFGF gene, and gene therapy DNA vector VTvaf17-NGF carrying the NGF gene for the increase of the expression level of BDNF, VEGFA, BFGF, and NGF proteins in mammalian tissues.

The change in the BDNF, VEGFA, BFGF, and NGF protein concentration in the rat's tibial muscle was assessed upon injection of a mixture of gene therapy vectors into this muscle.

Polyethyleneimine Transfection reagent cGMP grade in-vivo-jetPEI (Polyplus Transfection, France) was used as a transport system. Equimolar mixture of gene therapy DNA vectors was dissolved in sterile nuclease-free water. To obtain a gene construct, DNA-cGMP grade in-vivo-jetPEI complexes were prepared according to the manufacturer recommendations. The injectate volume was 0.05 ml with a total quantity of DNA equal to 100 μg. The solution injection was made into the rat's tibial muscle using the tunnel method with a 33 G needle to the depth of 2-3 mm.

The biopsy samples were taken on the 2nd day after the injection of the gene therapy DNA vectors. The biopsy sample was taken from muscle sites in the region of injection of a mixture of gene therapy DNA vectors carrying the genes BDNF, VEGFA, BFGF, and NGF (site I), gene therapy DNA vector VTvaf17 (placebo) (site II), as well as from the intact sites of another tibial muscle of animal (site III), using the skin biopsy device MAGNUM (BARD, USA). The biopsy sample site was preliminarily rinsed with sterile saline and anaesthetised with a lidocaine solution. The biopsy sample size was ca. 10 mm3, and the weight was approximately 11 mg. Each sample was placed in a buffer solution containing 50 mM of Tris-HCl, pH 7.6, 100 mM of NaCl, ImM of EDTA, and ImM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 17 (quantification of BDNF protein), Example 18 (quantification of VEGFA protein), Example 19 (quantification of BFGF protein), and Example 20 (quantification of NGF protein). Drawings resulting from the assay are shown in FIG. 22.

FIG. 22 demonstrates that there was an increase in BDNF, VEGFA, BFGF, and NGF protein concentration in the tibial muscle site of all rats (site I) where a mixture of gene therapy DNA vector VTvaf17-BDNF carrying the BDNF therapeutic gene, gene therapy DNA vector VTvaf17-VEGFA carrying the VEGFA therapeutic gene, gene therapy DNA vector VTvaf17-BFGF carrying the BFGF therapeutic gene, gene therapy DNA vector VTvaf17-NGF carrying the NGF therapeutic gene was injected compared to site II (placebo site) and site III (intact site). The obtained results show the efficiency of combined use of gene therapy DNA vector VTvaf17-BDNF, gene therapy DNA vector VTvaf17-VEGFA, gene therapy DNA vector VTvaf17-BFGF, and gene therapy DNA vector VTvaf17-NGF and practicability of their use for the increase of the expression level of therapeutic proteins in mammalian tissues.

### Example 30

Proof of the efficiency of gene therapy DNA vector VTvaf17-BFGF carrying the BFGF gene and practicability of its use in order to increase the expression level of BFGF protein in mammalian cells.

To confirm the efficiency of gene therapy DNA vector VTvaf17-BFGF carrying the BFGF gene, changes in mRNA accumulation of the BFGF therapeutic gene in BAOSMC bovine aortic smooth muscle cells (Genlantis) 48 hours after their transfection with gene therapy DNA vector VTvaf17-BFGF carrying the human BFGF gene were assessed.

BAOSMC bovine aortic smooth muscle cells were grown in Bovine Smooth Muscle Cell Growth Medium (Sigma B311F-500) with the addition of bovine serum up to 10% (Paneco, Russia). Transfection with gene therapy DNA vector VTvaf17-BFGF carrying the human BFGF gene and DNA vector VTvaf17 not carrying the human BFGF gene (reference), RNA extraction, reverse transcription reaction, PCR amplification, and data analysis were performed as described in Example 11. Bull/cow actin gene (ACT) listed in the GenBank database under number AH001130.2 was used as a reference gene. Positive control included amplicons from PCR on matrices represented by plasmids in known concentrations containing BFGF and ACT gene sequences. Negative control included deionised water. Real-time quantification of the PCR products, i.e. BFGF and ACT gene cDNAs obtained by amplification, was conducted using the Bio-Rad CFX Manager 2.1 software (Bio-Rad, USA).

Diagrams resulting from the assay are shown in FIG. 23.

FIG. 23 shows that the level of specific cDNA of human BFGF gene has grown massively as a result of transfection of BAOSMC bovine aortic smooth muscle cells with gene therapy DNA vector VTvaf17-BFGF, which confirms the ability of the vector to penetrate eukaryotic cells and express the BFGF gene at the mRNA level. The presented results confirm the practicability of use of gene therapy DNA vector VTvaf17-BFGF in order to increase the expression level of BFGF gene in mammalian cells.

### Example 31

Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 carrying the gene therapy DNA vector, and method of its production.

The construction of strain for the production of gene therapy DNA vector based on gene therapy DNA vector VTvaf17 carrying the therapeutic gene on an industrial scale selected from the group of the following genes: BDNF, VEGFA, BFGF, NGF, GDNF, NT3, CNTF, and IGF1 namely Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli SCS110-AF/VTvaf17-CNTF, or Escherichia coli strain SCS110-AF/VTvaf17-IGF1 carrying gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1, respectively, for its production allowing for antibiotic-free selection involves making electrocompetent cells of Escherichia coli strain SCS110-AF and subjecting these cells to electroporation with gene therapy DNA vector VTvaf17-BDNF, or gene therapy DNA vector VTvaf17-VEGFA, or gene therapy DNA vector VTvaf17-BFGF, or gene therapy DNA vector VTvaf17-NGF, or gene therapy DNA vector VTvaf17-GDNF, or gene therapy DNA vector VTvaf17-NT3, or gene therapy DNA vector VTvaf17-CNTF, or gene therapy DNA vector VTvaf17-IGF1. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

The obtained strains for production were included in the collection of the National Biological Resource Centre-Russian National Collection of Industrial Microorganisms (NBRC RNCIM), RF and NCIMB Patent Deposit Service, UK under the following registration numbers:

Escherichia coli strain SCS110-AF/VTvaf17-BDNF-registered at the Russian National Collection of Industrial Microorganisms under number B-13259, date of deposit 24.09.2018; INTERNATIONAL DEPOSITARY AUTHORITY No. NCIMB 43213, date of deposit 20.09.2018.

Escherichia coli strain SCS110-AF/VTvaf17-VEGFA-registered at the Russian National Collection of Industrial Microorganisms under number B-13344, date of deposit 22.11.2018; INTERNATIONAL DEPOSITARY AUTHORITY No. NCIMB 43289, date of deposit 22.11.2018.

Escherichia coli strain SCS110-AF/VTvaf17-BFGF-registered at the Russian National Collection of Industrial Microorganisms under number B-13278, date of deposit 16.10.2018; INTERNATIONAL DEPOSITARY AUTHORITY No. NCIMB 43303, date of deposit 13.12.2018.

Escherichia coli strain SCS110-AF/VTvaf17-NGF-registered at the Russian National Collection of Industrial Microorganisms under number B-13273, date of deposit 16.10.2018; INTERNATIONAL DEPOSITARY AUTHORITY No. NCIMB 43307, date of deposit 13.12.2018.

Escherichia coli strain SCS110-AF/VTvaf17-GDNF-registered at the Russian National Collection of Industrial Microorganisms under number B-13258, date of deposit 24.09.2018; INTERNATIONAL DEPOSITARY AUTHORITY No. NCIMB 43212, date of deposit 20.09.2018.

Escherichia coli strain SCS110-AF/VTvaf17-NT3-registered at the Russian National Collection of Industrial Microorganisms under number B-13339, date of deposit 22.11.2018; INTERNATIONAL DEPOSITARY AUTHORITY No. NCIMB 43285, date of deposit 22.11.2018.

Escherichia coli strain SCS110-AF/VTvaf17-CNTF-registered at the Russian National Collection of Industrial Microorganisms under number B-13276, date of deposit 16.10.2018; INTERNATIONAL DEPOSITARY AUTHORITY No. NCIMB 43298, date of deposit 13.12.2018.

Escherichia coli strain SCS110-AF/VTvaf17-IGF1-registered at the Russian National Collection of Industrial Microorganisms under number B-13274, date of deposit 16.10.2018; INTERNATIONAL DEPOSITARY AUTHORITY No. NCIMB 43304, date of deposit 13.12.2018.

### Example 32

The method for scaling up of the gene therapy DNA vector based on gene therapy DNA vector VTvaf17 carrying the therapeutic gene selected from the group of BDNF, VEGFA, BFGF, NGF, GDNF, NT3, CNTF, and IGF1 to an industrial scale.

To confirm the producibility and constructability on an industrial scale of gene therapy DNA vector VTvaf17-BDNF (SEQ ID No. 1), or VTvaf17-VEGFA (SEQ ID No. 2), or VTvaf17-BFGF (SEQ ID No. 3), or VTvaf17-NGF (SEQ ID No. 4), or VTvaf17-GDNF (SEQ ID No. 5), or VTvaf17-NT3 (SEQ ID No. 6), or VTvaf17-CNTF (SEQ ID No. 7), or VTvaf17-IGF1 (SEQ ID No. 8), large-scale fermentation of Escherichia coli strain SCS110-AF/VTvaf17-BDNF, or Escherichia coli SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1, each containing gene therapy DNA vector VTvaf17 carrying a region of the therapeutic gene, namely BDNF, or VEGFA, or BFGF, or NGF, or GDNF, or NT3, or CNTF, or IGF1. Each Escherichia coli strain SCS110-AF/VTvaf17-BDNF or Escherichia coli strain SCS110-AF/VTvaf17-VEGFA or Escherichia coli strain SCS110-AF/VTvaf17-BFGF or Escherichia coli strain SCS110-AF/VTvaf17-NGF or Escherichia coli strain SCS110-AF/VTvaf17-GDNF or Escherichia coli strain SCS110-AF/VTvaf17-NT3 or Escherichia coli strain SCS110-AF/VTvaf17-CNTF or Escherichia coli strain SCS110-AF/VTvaf17-IGF1 was produced on the basis of Escherichia coli strain SCS110-AF (Cell and Gene Therapy LLC, United Kingdom) as described in Example 31 by electroporation of competent cells of this strain with the gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 carrying the therapeutic gene, namely BDNF, VEGFA, BFGF, NGF, GDNF, NT3, CNTF, and IGF1 with further inoculation of transformed cells into agar plates (Petri dishes) with a selective medium containing yeastrel, peptone, and 6% sucrose, and selection of individual clones.

Fermentation of Escherichia coli SCS110-AF/VTvaf17-BDNF carrying gene therapy DNA vector VTvaf17-BDNF was performed in a 101 fermenter with subsequent extraction of gene therapy DNA vector VTvaf17-BDNF.

For the fermentation of Escherichia coli strain SCS110-AF/VTvaf17-BDNF, a medium was prepared containing (per 101 of volume): 100 g of tryptone and 50 g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvaf17-BDNF was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then, RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then, ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution and then gene therapy DNA vector VTvaf17-BDNF was eluted using a linear gradient of 25 mM TrisCl, pH 7.0, to obtain a solution of 25 mM TrisCl, pH 7.0, 1M NaCl, five times the volume of the column. The elution process was controlled by measuring optical density of the run-off solution at 260 nm. Chromatographic fractions containing gene therapy DNA vector VTvaf17-BDNF were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-BDNF were joined together and stored at −20° C. To assess the process reproducibility, the indicated processing operations were repeated five times. All processing operations for Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli SCS110-AF/VTvaf17-CNTF, or Escherichia coli strain SCS110-AF/VTvaf17-IGF1 were performed in a similar way.

The process reproducibility and quantitative characteristics of final product yield confirm the producibility and constructability of gene therapy DNA vector VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention, namely the construction of the gene therapy DNA vectors in order to increase the expression level of BDNF, VEGFA, BFGF, NGF, GDNF, NT3, CNTF, and IGF1 genes that combine the following properties:
I) The effectiveness of increase of expression of therapeutic genes in eukaryotic cells due to the obtained gene therapy vectors with limited size of vector part,
II) Possibility of safe use in gene therapy of human beings and animals due to the absence of regulatory elements representing the nucleotide sequences of viral genomes and antibiotic resistance genes in the gene therapy DNA vector,
III) Producibility and constructability in the strains on an industrial scale,
IV) as well as the purpose of the construction of strains carrying these gene therapy DNA vectors for the production of these gene therapy DNA vectors is achieved, which is supported by the following examples:
for I—Example 1, 2, 3, 4, 5; 6; 7; 8; 9; 10; 11; 12; 13; 14; 15; 16; 17; 18, 19, 20, 21, 22, 23, 24, 25, 26, 27, 28, 29, 30
for II—Example 1, 2, 3, 4, 5; 6; 7; 8; 9; 10; 11; 12; 13; 14; 15; 16; 17; 18, 19, 20, 21, 22, 23, 24, 25, 26, 27, 28, 29, 30
for III—Example 1, 2, 3, 4, 5; 6; 7; 8, 31, 32
for IV—Example 31, 32.

### INDUSTRIAL APPLICABILITY

All the examples listed above confirm the industrial applicability of the proposed gene therapy DNA vector based on gene therapy DNA vector VTvaf17 carrying the therapeutic gene selected from the group of BDNF, VEGFA, BFGF, NGF, GDNF, NT3, CNTF, and IGF1 genes in order to increase the expression level of these therapeutic genes, Escherichia coli strain SCS110-AF/VTvaf17-BDNF, or Escherichia coli strain SCS110-AF/VTvaf17-VEGFA, or Escherichia coli strain SCS110-AF/VTvaf17-BFGF, or Escherichia coli strain SCS110-AF/VTvaf17-NGF, or Escherichia coli strain SCS110-AF/VTvaf17-GDNF, or Escherichia coli strain SCS110-AF/VTvaf17-NT3, or Escherichia coli strain SCS110-AF/VTvaf17-CNTF, or Escherichia coli strain SCS110-AF/VTvaf17-IGF1 carrying gene therapy DNA vector, and method of its production on an industrial scale.

### LIST OF ABBREVIATIONS

VTvaf17-Gene therapy vector devoid of sequences of viral genomes and antibiotic resistance markers (vector therapeutic virus-antibiotic-free)
DNA-Deoxyribonucleic acid
cDNA-Complementary deoxyribonucleic acid
RNA-Ribonucleic acid
mRNA-Messenger ribonucleic acid
bp-base pair
PCR-Polymerase chain reaction
ml-millilitre, μl-microlitre
mm3-cubic millimetre
1-litre
μg-microgram
mg-milligram
g-gram
μM-micromol
mM-millimol
min-minute
s-second
rpm-rotations per minute
nm-nanometre
cm-centimetre
mW-milliwatt
RFU-Relative fluorescence unit
PBS-Phosphate buffered saline

### LIST OF REFERENCES

1. Aloe, L., Rocco, M. L., Bianchi, P., Manni, L., 2012. Nerve growth factor: from the early discoveries to the potential clinical use. J. Transl. Med. 10, 239
2. Autry A E, Monteggia L M. Brain-derived neurotrophic factor and neuropsychiatric disorders. Pharmacol Rev 2012; 64:238-258.
3. Bothwell, M., 2014. NGF, BDNF, NT3, and NT4. Handb. Exp. Pharmacol. 220, 3-15
4. Briand L A, Blendy J A. Molecular and genetic substrates linking stress and addiction. Brain Res 2010; 1314:219-234.
5 Chu, T.-H., Li, S.-Y., Guo, A., Wong, W.-M., Yuan, Q., Wu, W., 2009. Implantation of neurotrophic factor-treated sensory nerve graft enhances survival and axonal regeneration of motoneurons after spinal root avulsion. J. Neuropathol. Exp. Neurol. 68, 94-101
6. Connor B, Sun Y, von Hieber D, Tang S K, Jones K S, Maucksch C. AAV½-mediated BDNF gene therapy in a transgenic rat model of Huntington's disease. Gene Ther. 2016 March; 23 (3): 283-95.
7. Dijkhuizen, P. A., Pasterkamp, R. J., Hermens, W. T., de Winter, F., Giger, R. J., Verhaagen, J., 1998. Adenoviral vector-mediated gene delivery to injured rat peripheral nerve. J. Neurotrauma 15, 387-397
8. Draft Guideline on the quality, non-clinical and clinical aspects of gene therapy medicinal products, http://www.ema.europa.eu/docs/en_GB/document_library/Scientific_guideline/2015/05/WC50 0187020.pdf
9. Eggers, R., de Winter, F., Hoyng, S. a., Roet, K. C. D., Ehlert, E. M. Malessy, M. J. a, Verhaagen, J., Tannemaat, M. R., 2013. Lentiviral vector-mediated gradients of GDNF in the injured peripheral nerve: effects on nerve coil formation, Schwann cell maturation and myelination Deli M A, ed. PLOS One 8, e71076
10. Eggers, R.,Hendriks, W. T. J., Tannemaat, M. R., van Heerikhuize, J. J., Pool, C. W., Carlstedt, T. P., Zaldumbide, A., Hoeben, R. C., Boer, G. J., Verhaagen, J., 2008. Neuroregenerative effects of lentiviral vector-mediated GDNF expression in reimplanted ventral roots. Mol. Cell. Neurosci. 39, 105-117
11. Fine, E. G., Decosterd, I., Papaloïzos, M., Zurn, A. D., Aebischer, P., Papalozos, M., 2002. GDNF and NGF released by synthetic guidance channels support sciatic nerve regeneration across a long gap. Eur. J. Neurosci. 15, 589-601
12. Godinho, M. J., Teh, L., Pollett, M. A., Goodman, D., Hodgetts, S. I., Sweetman, I., Walters, M., Verhaagen, J., Plant, G. W., Harvey, A. R., 2013. Immunohistochemical, ultrastructural and functional analysis of axonal regeneration through peripheral nerve grafts containing Schwann cells expressing BDNF, CNTF or NT3. PLOS One 8, e69987
13. Gomez-Vargas Andrew, Gonzalo Hortelano, Michel Rathbone Ex-Vivo Gene Therapy Approach for Continuous Delivery of BDNF and Its Potential Use for Neurological Disorders Neurology April 2012, 78 (1 Supplement) IN6-1.008
14. Grinberg Y Y, Zitzow L A, Kraig R P. Intranasally administered IGF-1 inhibits spreading depression in vivo. Brain Res. 2017 Dec. 15; 1677:47-57
15. Guideline on the quality, non-clinical and clinical aspects of gene therapy medicinal Products EMA/CAT/80183/2014
16. Guillard E, Gueret G, Guillouet M, Vermeersch V, Rannou F, Giroux-Metges M A, Pennec J P. Alteration of muscle membrane excitability in sepsis: possible involvement of ciliary nervous trophic factor (CNTF). Cytokine. 2013 July; 63 (1): 52-57.
17. Haastert, K.,Lipokatic, E.,Fischer, M.,Timmer, M.,Grothe, C., 2006. Differentially promoted peripheral nerve regeneration by grafted Schwann cells over-expressing different FGF-2 isoforms. Neurobiol. Dis. 21, 138-153
18. Hornstein B D, Roman D, Arévalo-Soliz L M, Engevik M A, Zechiedrich L. Effects of Circular DNA Length on Transfection Efficiency by Electroporation into HeLa Cells. Ceña V, ed. PLOS ONE. 2016; 11 (12): e0167537.
19. Hu H, Lin H, Duan W, Cui C, Li Z, Liu Y, Wang W, Wen D, Wang Y, Li C. Intrathecal Injection of scAAV9-hIGF1 Prolongs the Survival of ALS Model Mice by Inhibiting the NF-KB Pathway. Neuroscience. 2018 Jun. 15; 381:1-10.
20. Hu, X., Cai, J., Yang, J., Smith, G. M., 2010. Sensory axon targeting is increased by NGF gene therapy within the lesioned adult femoral nerve. Exp. Neurol. 223, 153-165
21. Hu, Y., Leaver, S. G., Plant, G. W., Hendriks, W. T. J., Niclou, S. P., Verhaagen, J., Harvey, A. R., Cui, Q., 2005. Lentiviral-mediated transfer of CNTF to schwann cells within reconstructed peripheral nerve grafts enhances adult retinal ganglion cell survival and axonal regeneration. Mol. Ther. 11, 906-915
22. Jung G, Sun J, Petrowitz B, Riecken K, Kruszewski K, Jankowiak W, Kunst F, Skevas C, Richard G, Fehse B, Bartsch U. Genetically modified neural stem cells for a local and sustained delivery of neuroprotective factors to the dystrophic mouse retina. Stem Cells Transl Med. 2013 December; 2 (12): 1001-10.
23. Kale A, Joshi S, Pillai A, Naphade N, Raju M, Nasrallah H, Mahadik S P. Reduced cerebrospinal fluid and plasma nerve growth factor in drug-naïve psychotic patients. Schizophr Res. 2009 Aug. 25
24. Kiyota T, et al. FGF2 gene transfer restores hippocampal functions in mouse models of Alzheimer's disease and has therapeutic implications for neurocognitive disorders. Proc Natl Acad Sci USA. 2011; 108 (49): E1339-E1348.
25. Klimaschewski, L., Hausott, B., Angelov, D. N., 2013. The pros and cons of growth factors and cytokines in peripheral axon regeneration. Int. Rev. Neurobiol. 108, 137-171
26. Li L, Petrovsky N. Molecular mechanisms for enhanced DNA vaccine immunogenicity. Expert Rev Vaccines. 2016; 15 (3): 313-29
27. Lin L F, Doherty D H, Lile J D, Bektesh S, Collins F. GDNF: a glial cell line-derived neurotrophic factor for midbrain dopaminergic neurons. Science. 1993; 260:1130-1132.
28. Mairhofer J, Grabherr R. Rational vector design for efficient non-viral gene delivery: challenges facing the use of plasmid DNA. Mol Biotechnol. 2008.39 (2): 97-104
29. Mason, M. R. J., Tannemaat, M. R., Malessy, M. J. a, Verhaagen, J., 2011. Gene therapy for the peripheral nervous system: a strategy to repair the injured nerve? Curr. Gene Ther. 11, 75-89
30. Mishra N, Lata S, Deshmukh P, Kamat K, Surolia A, Banerjee T. Insulin signalling pathway protects neuronal cell lines by Sirt3 mediated IRS2 activation. Biofactors. 2018 May; 44 (3): 224-236.
31. Mitchelmore C, Gede L. Brain derived neurotrophic factor: epigenetic regulation in psychiatric disorders. Brain Res 2014; 1586:162-172.
32. Monteleone P, Maj M. Dysfunctions of leptin, ghrelin, BDNF and endocannabinoids in eating disorders: beyond the homeostatic control of food intake. Psychoneuroendocrinology 2013; 38; 312-330.
33. Newman, J. P., Verity, A. N., Hawatmeh, S., Fee, W. E. J., Terris, D. J., 1996b. Ciliary neurotrophic factors enhances peripheral nerve regeneration. Arch. Otolaryngol. Head Neck Surg. 122, 399-403.
34. Ogłodek EA, Just M J, Szromek A R, Araszkiewicz A. Melatonin and neurotrophins NT-3, BDNF, NGF in patients with varying levels of depression severity. Pharmacol Rep. 2016 October; 68 (5): 945-51.
35. Osborne A, Wang A XZ, Tassoni A, Widdowson P S, Martin K R. Design of a Novel Gene Therapy Construct to Achieve Sustained Brain-Derived Neurotrophic Factor Signalling in Neurons. Hum Gene Ther. 2018 July; 29 (7): 828-841.
36. Parkman H, Rao S, Reynolds J, et al. Neurotrophin-3 improves functional constipation. Am J Gastroenterol 2003; 98:1338-1347
37. Polyakova M, Stuke K, Schuemberg K, Mueller K, Schoenknecht P, Schroeter M L. BDNF as a biomarker for successful treatment of mood disorders: a systematic & quantitative meta-analysis. J Affect Disord 2015; 174:432-440.
38. Reflection paper on design modifications of gene therapy medicinal products during development/14 Dec. 2011 EMA/CAT/GTWP/44236/2009 Committee for advanced therapies
39. Revilla S, Ursulet S, Alvarez-López MJ, Castro-Freire M, Perpiñá U, García-Mesa Y, Bortolozzi A, Giménez-Llort L, Kaliman P, Cristòfol R, Sarkis C, Sanfeliu C. Lenti-GDNF gene therapy protects against Alzheimer's disease-like neuropathology in 3×Tg-AD mice and MC65 cells. CNS Neurosci Ther. 2014 November; 20 (11): 961-72.
40. Ruffini F, et al. Fibroblast growth factor-II gene therapy reverts the clinical course and the pathological signs of chronic experimental autoimmune encephalomyelitis in C57BL/6 mice. Gene Ther. 2001; 8 (16): 1207-1213.
41. S. A. Hoyng Fred De Winterab Sara Gnaviac Ralphde Boerb Lennard I. BoonaLaura M.Korversa Martijn R. Tannemaatad Martijn J. A. Malessyab Joost Verhaagen A comparative morphological, electrophysiological and functional analysis of axon regeneration through peripheral nerve autografts genetically modified to overexpress BDNF, CNTF, GDNF, NGF, NT3 or VEGFA. Experimental Neurology 261 (2014) 578-593
42. Sahenk Z, Galloway G, Clark K R, Malik V, Rodino-Klapac L R, Kaspar B K, Chen L, Braganza C, Montgomery C, Mendell J R. AAV1.NT-3 gene therapy for charcot-marie-tooth neuropathy. Mol Ther. 2014 March; 22 (3): 511-521.
43. Sahenk, Z, Seharaseyon, J.,Mendell, J. R., 1994. CNTF potentiates peripheral nerve regeneration. Brain Res. 655, 246-250
44. Santosa, K. B. Jesuraj, N. J., Viader, A., MacEwan, M., Newton, P., Hunter, D. A., Mackinnon, S. E., Johnson, P. J., 2013. Nerve allografts supplemented with schwann cells overexpressing glial-cell-line-derived neurotrophic factor. Muscle Nerve 47, 213-223
45. Sapieha P S, et al. Fibroblast growth factor-2 gene delivery stimulates axon growth by adult retinal ganglion cells after acute optic nerve injury. Mol Cell Neurosci. 2003; 24 (3): 656-672.
46. Shakhbazau, A., Mohanty, C., Shcharbin, D., Bryszewska, M., Caminade, A.-M., Majoral, J.-P., Alant, J., Midha, R., 2013. Doxycycline-regulated GDNF expression promotes axonal regeneration and functional recovery in transected peripheral nerve. J. Control. Release 172, 841-851
47. Sterne, G. D. Coulton, G. R., Brown, R. A., Green, C. J., Terenghi, G., 1997. Neurotrophin-3-enhanced nerve regeneration selectively improves recovery of muscle fibres expressing myosin heavy chains 2b. J. Cell Biol. 139, 709-715
48. Storkebaum E., Lambrechts D., Carmeliet P. VEGFA: once regarded as a specific angiogenic factor, now implicated in neuroprotection//Bioessays. 2004. T. 26. No. 9. P. 943-954.
49. Sun D, et al. Basic fibroblast growth factor-enhanced neurogenesis contributes to cognitive recovery in rats following traumatic brain injury. Exp Neurol. 2009; 216 (1): 56-65.
50. Tannemaat, M. R., Eggers, R., Hendriks, W. T., De Ruiter, G. C. W., Van Heerikhuize, J. J., Pool, C. W., Malessy, M. J. a, Boer, G. J., Verhaagen, J., 2008. Differential effects of lentiviral vector-mediated overexpression of nerve growth factor and glial cell line-derived neurotrophic factor on regenerating sensory and motor axons in the transected peripheral nerve. Eur. J. Neurosci. 28, 1467-1479
51. Utley, D. S., Lewin, S. L., Cheng, E. T., Verity, A. N., Sierra, D., Terris, D. J., 1996. Brain-derived neurotrophic factor and collagen tubulisation enhance functional recovery after peripheral nerve transection and repair. Arch. Otolaryngol. Head Neck Surg. 122, 407-413
52. Wan G, Gómez-Casati M E, Gigliello A R, Liberman M C, Corfas G. Neurotrophin-3 regulates ribbon synapse density in the cochlea and induces synapse regeneration after acoustic trauma. Elife. 2014 Oct. 20; 3.
53. Wennberg A MV, Hagen C E, Machulda M M, Hollman J H, Roberts R O, Knopman D S, Petersen R C, Mielke M M. The association between peripheral total IGF-1, IGFBP-3, and IGF-1/IGFBP-3 and functional and cognitive outcomes in the Mayo Clinic Study of Aging. Neurobiol Aging. 2018 June; 66:68-74.
54. Wood, M. D., Kim, H., Bilbily, A., Kemp, S. W. P., Lafontaine, C., Gordon, T., Shoichet, M. S., Borschel, G. H., 2012. GDNF released from microspheres enhances nerve regeneration after delayed repair. Muscle Nerve 46, 122-124
55. Wood, M. D., Moore, A. M., Hunter, D. A., Tuffaha, S., Borschel, G. H., Mackinnon, S. E., Sakiyama-Elbert, S. E., 2009. Affinity-based release of glial-derived neurotrophic factor 592 S. A. Hoyng et al./Experimental Neurology 261 (2014) 578-593
56. Wood, M. D., Gordon, T., Kim, H., Szynkaruk, M., Phua, P., Lafontaine, C., Kemp, S. W., Shoichet, M. S., Borschel, G. H., 2013. Fibrin gels containing GDNF microspheres increase axonal regeneration after delayed peripheral nerve repair. Regen. Med. 8, 27-37
57. Yalvac M E, Amornvit J, Chen L, Shontz K M, Lewis S, Sahenk Z. AAV1.NT-3 gene therapy increases muscle fibre diameter through activation of mTOR pathway and metabolic remodelling in a CMT mouse model. Gene Ther. 2018 April; 25 (2): 129-138.
58. Yalvac M E, Arnold W D, Braganza C, Chen L, Mendell J R, Sahenk Z. AAV1.NT-3 gene therapy attenuates spontaneous autoimmune peripheral polyneuropathy. Gene Ther. 2016 January; 23 (1): 95-102.
59. Zhou Z, Shi P, Cai J. The effects of exogenous human glial cell line-derived neurotrophic factor on functional regeneration of severed femoral nerve and its underlying mechanisms. J Cell Biochem. 2018; 1-7.
60. Molecular Biology, 2011, Vol. 45, No. 1, p. 44-55

What is claimed is:

1.-22. (canceled)

23. A gene therapy DNA vector based on a gene therapy DNA vector VTvaf17 for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, including injuries, neurodegenerative diseases, diabetic neuropathy, conditions resulting in damages to central nervous system, conditions following acute ischemia, for improvement of cognitive functions, as neuroprotective action against oxidative stress, wherein the gene therapy DNA vector has a coding region of a therapeutic gene, selected from BDNF, or VEGFA, or BFGF, or NGF, or GDNF, or NT3, or CNTF, or IGF1, cloned to the gene therapy DNA vector VTvaf17 resulting in a gene therapy DNA vector VTvaf17-BDNF that has a nucleotide sequence SEQ ID No. 1, or resulting in a gene therapy DNA vector VTvaf17-VEGFA that has a nucleotide sequence SEQ ID No. 2, or resulting in a gene therapy DNA vector VTvaf17-BFGF that has a nucleotide sequence SEQ ID No. 3, or resulting in a gene therapy DNA vector VTvaf17-NGF that has a nucleotide sequence SEQ ID No. 4, or resulting in a gene therapy DNA vector VTvaf17-GDNF that has a nucleotide sequence SEQ ID No. 5, or resulting in a gene therapy DNA vector VTvaf17-NT3 that has a nucleotide sequence SEQ ID No. 6, or resulting in a gene therapy DNA vector VTvaf17-CNTF that has a nucleotide sequence SEQ ID No. 7, or resulting in a gene therapy DNA vector VTvaf17-IGF1 that has a nucleotide sequence SEQ ID No. 8 respectively.

24. The gene therapy DNA vector based on the gene therapy DNA vector VTvaf17 carrying BDNF, or VEGFA, or BFGF, or NGF, or GDNF, or NT3, or CNTF, or IGF1 therapeutic gene as per claim 23, said gene therapy DNA vectors being unique due to a fact that each of the gene therapy DNA vectors: VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 uses nucleotide sequences that are not antibiotic resistance genes, virus genes, or regulatory elements of viral genomes being used as structure elements, which ensures a safe use for a gene therapy in humans and animals.

25. A method of gene therapy DNA vector production based on the gene therapy DNA vector VTvaf17 carrying BDNF, or VEGFA, or BFGF, or NGF, or GDNF, or NT3, or CNTF, or IGF1 therapeutic gene as per claim 23 that involves obtaining each of gene therapy DNA vectors: VTvaf17-BDNF, or VTvaf17-VEGFA, or VTvaf17-BFGF, or VTvaf17-NGF, or VTvaf17-GDNF, or VTvaf17-NT3, or VTvaf17-CNTF, or VTvaf17-IGF1 as follows: a coding region of BDNF, or VEGFA, or BFGF, or NGF, or GDNF, or NT3, or CNTF, or IGF1 therapeutic gene is cloned to the gene therapy DNA vector VTvaf17, and the gene therapy DNA vector VTvaf17-BDNF, SEQ ID No. 1, or VTvaf17-VEGFA, SEQ ID No. 2, or VTvaf17-BFGF, SEQ ID No. 3, or VTvaf17-NGF, SEQ ID No. 4, or VTvaf17-GDNF, SEQ ID No. 5, or VTvaf17-NT3, SEQ ID No. 6, or VTvaf17-CNTF, SEQ ID No. 7, or VTvaf17-IGF1, SEQ ID No. 8, respectively, is obtained, while the coding region of the BDNF, or VEGFA, or BFGF, or NGF, or GDNF, or NT3, or CNTF, or IGF1 therapeutic gene is obtained by isolating total RNA from a human biological tissue sample followed by a reverse transcription reaction and a PCR amplification using the obtained oligonucleotides and cleaving the amplification product by corresponding restriction endonucleases, while cloning to the gene therapy DNA vector VTvaf17 is performed by NheI and HindIII, restriction sites, while a selection is performed without antibiotics,
at the same time, following oligonucleotides are used during a gene therapy DNA vector VTvaf17-BDNF, SEQ ID No. 1 production for the reverse transcription reaction and the PCR amplification:
BDNF_F GGATCCGCCACCATGACCATCCTTTTCCTTACTATG,
BDNF_R AGGGAATTCCTATCTTCCCCTTTTAATGGTC, and the cleaving of the amplification product and cloning of the coding region of BDNF gene to the gene therapy DNA vector VTvaf17 is performed by BamHI and EcoRI restriction endonucleases,
at the same time, following oligonucleotides are used during a gene therapy DNA vector VTvaf17-VEGFA, SEQ ID No. 2 production for the reverse transcription reaction and the PCR amplification:
VEGFA_F GGGGGATCCACCATGACGGACAGACAGACAGACACCGC,
VEGFA_R TTTGGATCCACCATGAACTTTCTGCTGTCTTGGGTGC, and the cleaving of the amplification product and cloning of the coding region of VEGFA gene to the gene therapy DNA vector VTvaf17 is performed by BamHI and HindIII restriction endonucleases,
at the same time, following oligonucleotides are used during a gene therapy DNA vector VTvaf17-BFGF, SEQ ID No. 3 production for the reverse transcription reaction and the PCR amplification:
BFGF_F GAGGAAGCTTCCACCATGGTGGGTGTGGGGGGTGGAGATG,
BFGF_R GAGGGAATTCTCAGCTCTTAGCAGACATTGGAAGA, and the cleaving of the amplification product and cloning of the coding region of BFGF gene to the gene therapy DNA vector VTvaf17 is performed by HindIII and EcoRI restriction endonucleases,
at the same time, following oligonucleotides are used during a gene therapy DNA vector VTvaf17-NGF, SEQ ID No. 4 production for the reverse transcription reaction and the PCR amplification:
NGF_F TTTGTCGACCACCATGTCCATGTTGTTCTACACTCTGATC,
NGF_R AATGGTACCTCAGGCTCTTCTCACAGCCTTCC, and the cleaving of the amplification product and cloning of the coding region of NGF gene to the gene therapy DNA vector VTvaf17 is performed by SaII and KpnI restriction endonucleases,
at the same time, following oligonucleotides are used during a gene therapy DNA vector VTvaf17-GDNF, SEQ ID No. 5 production for the reverse transcription reaction and the PCR amplification:
GDNF_F GGGGGATCCACCATGCAGTCTTTGCCTAACAGCAATGG,
GDNF_R TTTAAGCTTTCAGATACATCCACACCTTTTAGCG, and the cleaving of the amplification product and cloning of the coding region of GDNF gene to the gene therapy DNA vector VTvaf17 is performed by BamHI and HindIII restriction endonucleases,
at the same time, following oligonucleotides are used during a gene therapy DNA vector VTvaf17-NT3, SEQ ID No. 6 production for the reverse transcription reaction and the PCR amplification:
NT3_F AGGATCCACCATGGTTACTTTTGCCACGATC,
NT3_R TATAAGCTTTCATGTTCTTCCGATTTTTCTC, and the cleaving of the amplification product and cloning of the coding region of NT3 gene to the gene therapy DNA vector VTvaf17 is performed by BamHI and HindIII restriction endonucleases,
at the same time, following oligonucleotides are used during a gene therapy DNA vector VTvaf17-CNTF, SEQ ID No. 7 production for the reverse transcription reaction and the PCR amplification:
CNTF_F TTTGTCGACCACCATGGCTTTCACAGAGCATTCACC,
CNTF_R AATGGTACCTACATTTTCTTGTTGTTAGCAATATAATGG, and the cleaving of the amplification product and cloning of the coding region of CNTF gene to the gene therapy DNA vector VTvaf17 is performed by BamHII and HindIII restriction endonucleases,
at the same time, following oligonucleotides are used during gene therapy DNA vector VTvaf17-IGF1, SEQ ID No. 8 production for the reverse transcription reaction and the PCR amplification:
IGF1_F TTTGTCGACCACCATGGGAAAAATCAGCAGTCTTCC,
IGF1_R AATGGTACCTACTTGCGTTCTTCAAATGTACTTCC, and the cleaving of the amplification product and cloning of the coding region of IGF1 gene to the gene therapy DNA vector VTvaf17 is performed by SaII and KpnI restriction endonucleases.

26. A method of use of the gene therapy DNA vector based on the gene therapy DNA vector VTvaf17 carrying BDNF, or VEGFA, or BFGF, or NGF, or GDNF, or NT3, or CNTF, or IGF1 therapeutic gene as per claim 23 for treatment of diseases associated with disorders of central and peripheral nervous system function, disorders of neurogenesis, for stimulation of neuronal growth, including for improvement of potential of cell therapy and allogeneic grafts, for improvement of neurogenesis, including for treatment of diseases, including injuries, neurodegenerative diseases, diabetic neuropathy, conditions resulting in damages to central nervous system, conditions following acute ischemia, for improvement of cognitive functions, as neuroprotective action against oxidative stress that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from a group of the constructed gene therapy DNA vectors carrying therapeutic genes based on the gene therapy DNA vector VTvaf17 and injection of autologous cells of a patient or an animal transfected by the selected gene therapy DNA vector carrying the therapeutic gene based on the gene therapy DNA vector VTvaf17 or the several selected gene therapy DNA vectors carrying the therapeutic genes based on the gene therapy DNA vector VTvaf17 from the constructed gene therapy DNA vectors carrying therapeutic genes based on the gene therapy DNA vector VTvaf17 into organs and tissues of the same patient or the animal and the injection of the selected gene therapy DNA vector carrying therapeutic gene based on the gene therapy DNA vector VTvaf17 or the several selected gene therapy DNA vectors carrying the therapeutic genes based on the gene therapy DNA vector VTvaf17 from a group of the constructed gene therapy DNA vectors carrying therapeutic genes based on the gene therapy DNA vector VTvaf17 into the organs and the tissues of the same patient or the animal, or the combination of the indicated methods.
